(19) United States
(12) Patent Application Publication
(10) Pub. No.: US 2026/0334824 A1
(43) Pub. Date: Oct. 29, 2026

(54) SYSTEMS, DEVICES, AND METHODS FOR ANALYTE MONITORING

(71) Applicant: ABBOTT DIABETES CARE INC., Alameda, CA (US)

(72) Inventors: Matthew Simmons, Pleasanton, CA (US); Steven T. Mitchell, Pleasant Hill, CA (US)

(21) Appl. No.: 19/726,661

(22) Filed: Jun. 30, 2026

Related U.S. Application Data
(63) Continuation of application No. 18/092,579, filed on Jan. 3, 2023, now Pat. No. 12,702,332.
(60) Provisional application No. 63/296,262, filed on Jan. 4, 2022.

(51) Int. Cl. A61B 5/145; A61B 5/00

(52) U.S. Cl. CPC A61B 5/14546; A61B 5/14503; A61B 5/6848

(57) ABSTRACT

Systems, devices and methods are provided for inserting at least a portion of an in vivo analyte sensor for sensing an analyte level in a bodily fluid of a subject. In particular, disclosed herein are various embodiments of applicators, and components thereof, designed to reduce trauma to tissue of a sensor insertion site and to increase the likelihood of a successful sensor insertion.

### CROSS-REFERENCE TO RELATED APPLICATIONS

This application is a continuation of U.S. application Ser. No. 18/092,579, filed Jan. 3, 2023, which claims priority to U.S. Provisional Application No. 63/296,262, filed Jan. 4, 2022, both of which are herein expressly incorporated by reference in their entireties for all purposes.

### FIELD

The subject matter described herein relates generally to systems, devices, and methods for in vivo analyte monitoring.

### BACKGROUND

The detection and/or monitoring of analyte levels, such as glucose, ketones, lactate, oxygen, hemoglobin AIC, or the like, can be vitally important to the overall health of a person, particularly for an individual having diabetes. Patients suffering from diabetes mellitus can experience complications including loss of consciousness, cardiovascular disease, retinopathy, neuropathy, and nephropathy. Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

Devices have been developed for the automatic monitoring of analyte(s), such as glucose, in bodily fluid such as in the blood stream or in interstitial fluid (“ISF”), or other biological fluid. Some of these analyte measuring devices are configured so that at least a portion of the devices are positioned below a skin surface of a user, e.g., in a blood vessel or in the subcutaneous tissue of a user, so that the monitoring is accomplished in vivo.

With the continued development of analyte monitoring devices and systems, there is a need for such analyte monitoring devices, systems, and methods, as well as for processes for manufacturing analyte monitoring devices and systems that are cost effective, convenient, and provide discreet monitoring to encourage frequent analyte monitoring to improve glycemic control. Additionally, there is a need for such analyte monitoring devices, systems, and methods that reduce pain and trauma associated with analyte monitoring and testing.

While current sensors can be convenient for users, they are also susceptible to malfunctions. These malfunctions can be caused by user error, lack of proper training, poor user coordination, overly complicated procedures, physiological responses to the inserted sensor, and other issues. This can be particularly true for analyte monitoring systems having sensors used to measure an analyte level in ISF, and which are inserted using sharps (also known as “introducers” or “needles”). In addition, some prior art systems may utilize sharps that can create trauma to surrounding tissue at the sensor insertion site, which can lead to inaccurate analyte level measurements. These challenges and others described herein can lead to a failure to properly monitor the patient's analyte level.

Thus, a need exists for more reliable sensor insertion devices, systems and methods, that are easy to use by the patient, less prone to error, and which reduce trauma to an insertion site.

### SUMMARY

Provided herein are example embodiments of systems, devices and methods for the assembly and use of an applicator and a sensor control device of an in vivo analyte monitoring system. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. In certain embodiments, the applicator comprises a sensor having a sharpened or V-shaped tip portion and/or a sharp having a V-shaped cross-sectional area. The V-shaped sensors and V-shaped sharps described herein are configured to minimize skin penetration and puncture wound size during sensor insertion. In some embodiments, the applicator is sharpless and, thus, does not comprise a sharp or sharp module. In the sharpless applicator embodiments described herein, the sensor may be configured to provide the necessary physical characteristics to increase insertion effectiveness.

The applicator can be used to position the sensor control device on a human body with a sensor in contact with the wearer's bodily fluid. The embodiments provided herein are improvements to prevent or reduce the likelihood that a sensor elicits an adverse physiological response. Other improvements and advantages are provided as well. The various configurations of these devices are described in detail by way of embodiments which are only examples.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIGS. 3A to 3G are progressive views of an example embodiment of the assembly and application of the system of FIG. 1 incorporating a two-piece architecture.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 5 is a proximal perspective view depicting an example embodiment of a tray with sterilization lid coupled.

FIG. 6A is a proximal perspective cutaway view depicting an example embodiment of a tray with sensor delivery components.

FIG. 6B is a proximal perspective view depicting sensor delivery components.

FIG. 7A is side view depicting an example embodiment of a housing.

FIG. 7B is a perspective view depicting an example embodiment of a distal end of a housing.

FIG. 7C is a side cross-sectional view depicting an example embodiment of a housing.

FIG. 8A is a side view depicting an example embodiment of a sheath.

FIG. 8B is a perspective view depicting an example embodiment of a proximal end of a sheath.

FIG. 8C is a close-up perspective view depicting an example embodiment of a distal side of a detent snap of a sheath.

FIG. 8D is a side view depicting an example embodiment of features of a sheath.

FIG. 8E is an end view of an example embodiment of a proximal end of a sheath.

FIG. 9A is a proximal perspective view depicting an example embodiment of a device carrier.

FIG. 9B is a distal perspective view depicting an example embodiment of a device carrier.

FIG. 10 is a proximal perspective view of an example embodiment of a sharp carrier.

FIG. 11 is a side cross-section depicting an example embodiment of a sharp carrier.

FIGS. 12A to 12B are top and bottom perspective views, respectively, depicting an example embodiment of a sensor module.

FIGS. 13A and 13B are perspective views depicting an example embodiment of a sensor connector.

FIG. 14A is a perspective view depicting an example embodiment of a sensor.

FIG. 14B is a side view of an exemplary sensor, in accordance with one embodiment of the disclosed subject matter.

FIG. 14C is a side view of an exemplary sensor and a callout view of a tip portion of an exemplary sensor, in accordance with one embodiment of the disclosed subject matter.

FIG. 15A is a perspective view depicting an example embodiment of a sharp module.

FIG. 15B is a perspective view depicting an example embodiment of a sharp module, and a callout view of the distal tip thereof.

FIG. 15C is a close-up side perspective view depicting the distal tip of the sharp module illustrated in FIG. 15B.

FIG. 15D is a cross-sectional view of a sharp embodiment for the sharp module illustrated in FIG. 15B.

FIG. 15E is a close-up perspective view of a sharp in accordance with one embodiment of the disclosed subject matter.

FIG. 15F is a close-up side view of a sharp in accordance with one embodiment of the disclosed subject matter.

FIG. 15G is a cross-sectional view of a sharp in accordance with one embodiment of the disclosed subject matter.

FIG. 15H is a cross-sectional view of the sharp depicted in FIG. 15G, further comprising a sensor in accordance with one embodiment of the disclosed subject matter.

FIG. 15I is a cross-sectional view of a sharp in accordance with one embodiment of the disclosed subject matter.

FIG. 15J is a cross-sectional view of the sharp depicted in FIG. 15I, further comprising a sensor in accordance with one embodiment of the disclosed subject matter.

FIG. 15K is a partial perspective view of a sharp module in accordance with one embodiment of the disclosed subject matter, and a callout view of the distal tip thereof.

FIG. 15L is a close-up perspective view of the distal tip of a sharp module comprising a sensor, in accordance with one embodiment of the disclosed subject matter.

FIG. 16A is a cross-sectional view of a sharpless applicator, in accordance with one embodiment of the disclosed subject matter.

FIGS. 16B to 16H are cross-sectional views of the sharpless applicator depicted in FIG. 16A during various stages of operation.

FIG. 16I is a cross-sectional view depicting an example embodiment of a sharpless applicator illustrated in FIG. 16A.

FIG. 16J and 16J-1 are cross-sectional and call-out close-up views, respectively, depicting an example embodiment of a guide-sensor control device assembly.

FIG. 16K is a cross-sectional view depicting an example embodiment of a sharpless applicator.

FIG. 17A-17A-2 are cross-sectional and callout views of an example embodiment of a sharpless applicator, in accordance with one embodiment of the disclosed subject matter.

FIGS. 17B to 17D are cross-sectional views of the sharpless applicator depicted in FIG. 17A during various stages of operation.

FIGS. 18A to 18C are cross-sectional views of a powered sharpless applicator during various stages of operation.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

As mentioned, a number of embodiments of systems, devices, and methods are described herein that provide for improved sensor insertion devices for use with in vivo analyte monitoring systems. Several embodiments of the present disclosure also provide for improved insertion sharp modules and sensors. Many embodiments of the present disclosure are designed to: improve the method of sensor insertion with respect to in vivo analyte monitoring systems, minimize trauma to an insertion site during a sensor insertion process, and reduce overall interference with sensor performance. Some embodiments, for example, include an applicator having a sharp with a V-shaped cross-sectional area comprising a vertex. This can allow for a smaller skin penetration and, thus, create a smaller wound with less trauma at the insertion site, which can reduce the chance of early signal attenuation (“ESA”). In other embodiments, the applicator includes a sensor with a V-shaped tip portion which can minimize trauma to an inserting site during a sensor insertion process and improve the likelihood of a successful sensor insertion. In still other embodiments, a sharpless applicator is configured to penetrate the skin utilizing a sensor having a sharpened or pointed V-shaped tip portion in order to reduce trauma to an insertion site. In some sharpless applicator embodiments, the design utilizes the deformation or “tenting” of the skin during insertion as a source of potential energy to provide for a more effective insertion. In sum, these embodiments can improve the likelihood of a successful sensor insertion and reduce the amount of trauma at the insertion site, to name a few advantages.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary In Vivo Analyte Monitoring System

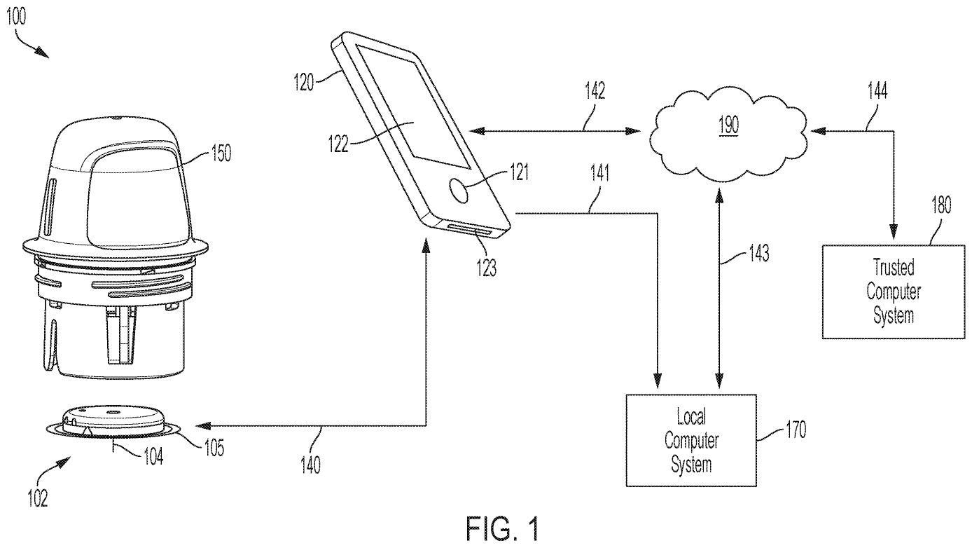

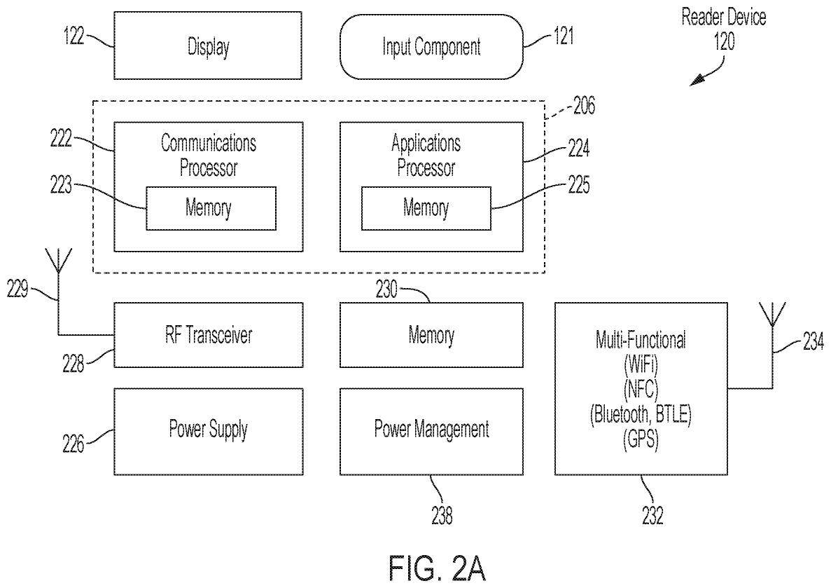

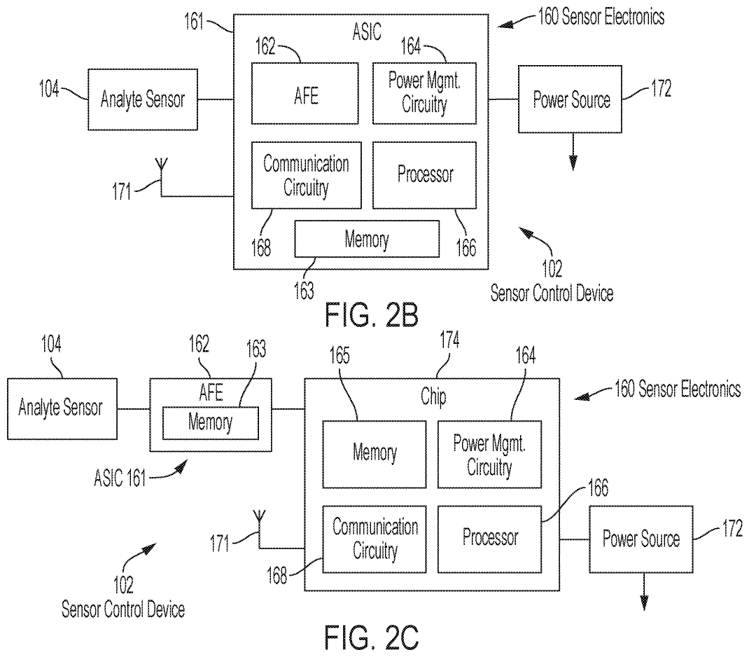

FIG. 1 is a conceptual diagram depicting an example embodiment of an analyte monitoring system 100 that includes a sensor applicator 150, a sensor control device 102, and a reader device 120. Sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121 and the device battery can be recharged using power port 123. More detail about reader device 120 is set forth with respect to FIG. 2A below. Reader device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy, Wi-Fi or others. Local computer system 170 can communicate via communications path 143 with a network 190 similar to how reader device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Exemplary Reader Device

FIG. 2A is a block diagram depicting an example embodiment of a reader device configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Exemplary Sensor Control Devices

FIGS. 2B and 2C are block diagrams depicting example embodiments of sensor control device 102 having analyte sensor 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Exemplary Assembly Processes for Sensor Control Devices

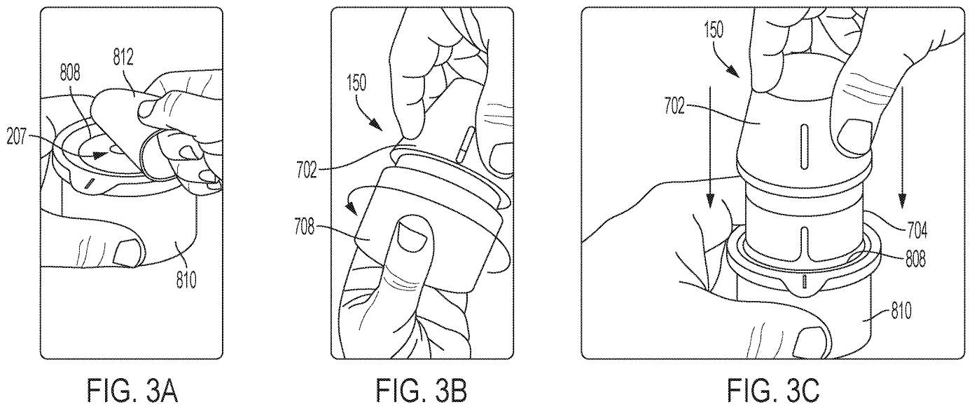

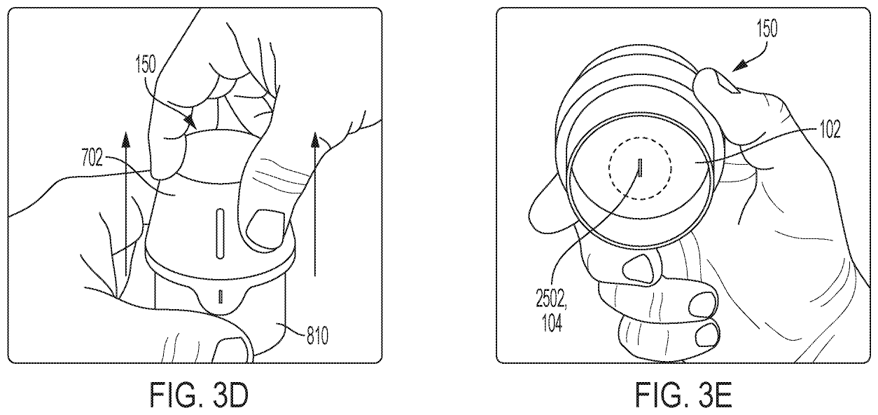

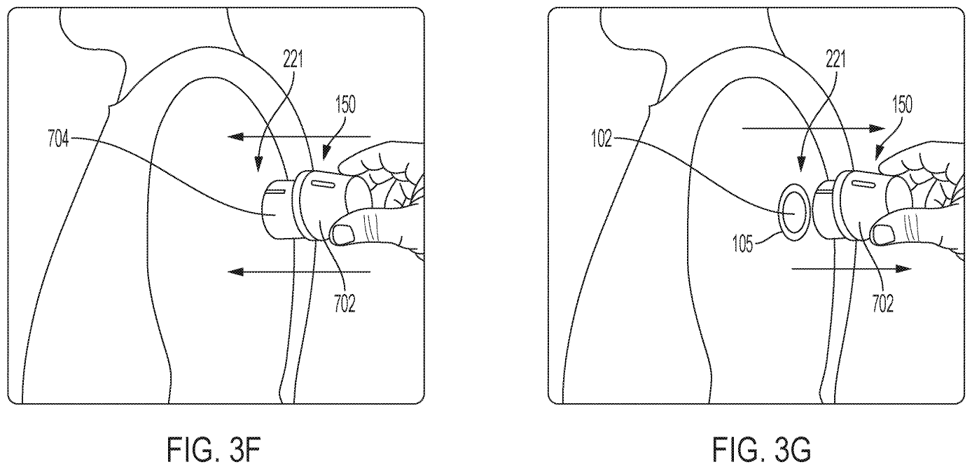

According to some embodiments, the components of sensor control device 102 can be acquired by a user in multiple packages requiring final assembly by the user before delivery to an appropriate user location. FIGS. 3A-3E depict an example embodiment of an assembly process for sensor control device 102 by a user, including preparation of separate components before coupling the components in order to ready the sensor for delivery. In other embodiments, components of the sensor control device 102 and applicator 150 can be acquired by a user in a single package. FIGS. 3F-3G depict an example embodiment of delivery of sensor control device 102 to an appropriate user location by selecting the appropriate delivery location and applying device 102 to the location.

FIG. 3A depicts a sensor container or tray 810 that has a removable lid 812. The user prepares the sensor tray 810 by removing the lid 812, which acts as a sterile barrier to protect the internal contents of the sensor tray 810 and otherwise maintain a sterile internal environment. Removing the lid 812 exposes a platform 808 positioned within the sensor tray 810, and a plug assembly 207 (partially visible) is arranged within and otherwise strategically embedded within the platform 808. The plug assembly 207 includes a sensor module (not shown) and a sharp module (not shown). The sensor module carries the sensor 104 (FIG. 1), and the sharp module carries an associated sharp used to help deliver the sensor 104 transcutaneously under the user's skin during application of the sensor control device 102 (FIG. 1).

FIG. 3B depicts the sensor applicator 150 and the user preparing the sensor applicator 150 for final assembly. The sensor applicator 150 includes a housing 702 sealed at one end with an applicator cap 708. In some embodiments, for example, an O-ring or another type of sealing gasket may seal an interface between the housing 702 and the applicator cap 708. In at least one embodiment, the O-ring or sealing gasket may be molded onto one of the housing 702 and the applicator cap 708. The applicator cap 708 provides a barrier that protects the internal contents of the sensor applicator 150. In particular, the sensor applicator 150 contains an electronics housing (not shown) that retains the electrical components for the sensor control device 102 (FIG. 1), and the applicator cap 708 may or may not maintain a sterile environment for the electrical components. Preparation of the sensor applicator 150 includes uncoupling the housing 702 from the applicator cap 708, which can be accomplished by unscrewing the applicator cap 708 from the housing 702. The applicator cap 708 can then be discarded or otherwise placed aside.

FIG. 3C depicts the user inserting the sensor applicator 150 into the sensor tray 810. The sensor applicator 150 includes a sheath 704 configured to be received by the platform 808 to temporarily unlock the sheath 704 relative to the housing 702, and also temporarily unlock the platform 808 relative to the sensor tray 810. Advancing the housing 702 into the sensor tray 810 results in the plug assembly 207 (FIG. 3A) arranged within the sensor tray 810, including the sensor and sharp modules, being coupled to the electronics housing arranged within the sensor applicator 150.

In FIG. 3D, the user removes the sensor applicator 150 from the sensor tray 810 by proximally retracting the housing 702 with respect to the sensor tray 810.

FIG. 3E depicts the bottom or interior of the sensor applicator 150 following removal from the sensor tray 810 (FIGS. 3A and 3C). The sensor applicator 150 is removed from the sensor tray 810 with the sensor control device 102 fully assembled therein and positioned for delivery to the target monitoring location. As illustrated, a sharp 2502 extends from the bottom of the sensor control device 102 and carries a portion of the sensor 104 within a hollow or recessed portion thereof. The sharp 2502 is configured to penetrate the skin of a user and thereby place the sensor 104 into contact with bodily fluid.

FIGS. 3F and 3G depict example delivery of the sensor control device 102 to a target monitoring location 221, such as the back of an arm of the user. FIG. 3F shows the user advancing the sensor applicator 150 toward the target monitoring location 221. Upon engaging the skin at the target monitoring location 221, the sheath 704 collapses into the housing 702, which allows the sensor control device 102 (FIGS. 3E and 3G) to advance into engagement with the skin. With the help of the sharp 2502 (FIG. 3E), the sensor 104 (FIG. 3E) is advanced transcutaneously into the patient's skin at the target monitoring location 221.

FIG. 3G shows the user retracting the sensor applicator 150 from the target monitoring location 221, with the sensor control device 102 successfully attached to the user's skin. The adhesive patch 105 (FIG. 1) applied to the bottom of sensor control device 102 adheres to the skin to secure the sensor control device 102 in place. The sharp 2502 (FIG. 3E) is automatically retracted when the housing 702 is fully advanced at the target monitoring location 221, while the sensor 104 (FIG. 3E) is left in position to measure analyte levels.

According to some embodiments, system 100, as described with respect to FIGS. 3A-3G and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly. Further details regarding embodiments of applicators, their components, and variants thereof, are described in U.S. Patent Publication Nos. 2013/0150691, 2016/0331283, and 2018/0235520, all of which are incorporated by reference herein in their entireties and for all purposes.

### Example Embodiment of Sensor Applicator Device

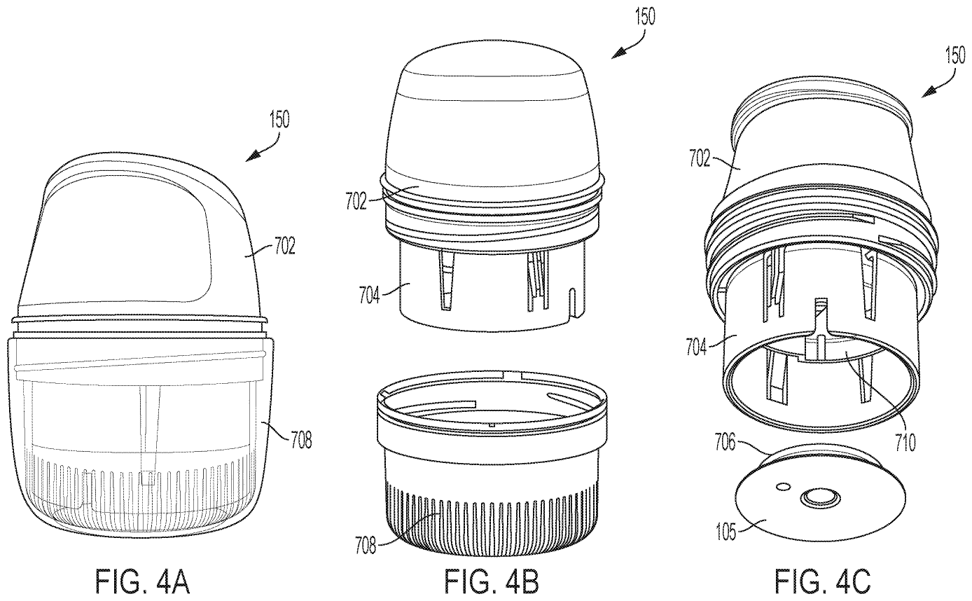

FIG. 4A is a side view depicting an example embodiment of an applicator device 150 coupled with screw cap 708. This is one example of how applicator 150 is shipped to and received by a user, prior to assembly by the user with a sensor. In other embodiments, applicator 150 can be shipped to the user with the sensor and sharp contained therein. FIG. 4B is a side perspective view depicting applicator 150 and cap 708 after being decoupled. FIG. 4C is a perspective view depicting an example embodiment of a distal end of an applicator device 150 with electronics housing 706 and adhesive patch 105 removed from the position they would have retained within device carrier 710 of sheath 704, when cap 708 is in place.

### Exemplary Tray and Sensor Module Assembly

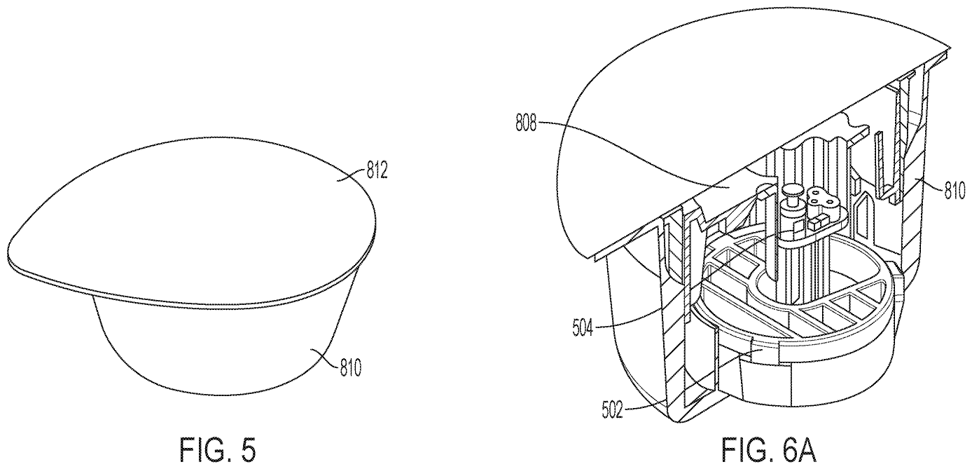

FIG. 5 is a proximal perspective view depicting an example embodiment of a tray 810 with sterilization lid 812 removably coupled thereto, which may be representative of how the package is shipped to and received by a user prior to assembly.

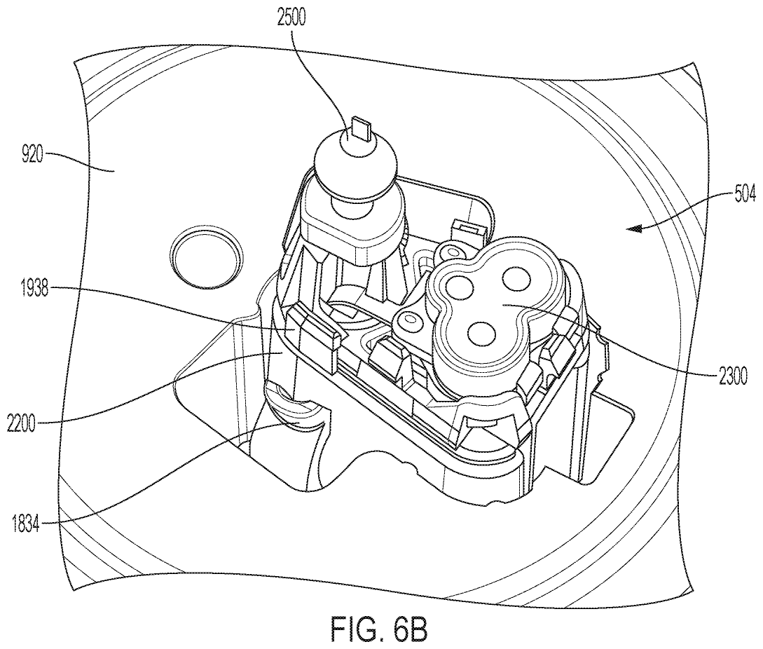

FIG. 6A is a proximal perspective cutaway view depicting sensor delivery components within tray 810. Platform 808 is slidably coupled within tray 810. Desiccant 502 is stationary with respect to tray 810. Sensor module 504 is mounted within tray 810.

FIG. 6B is a proximal perspective view depicting sensor module 504 in greater detail. Here, retention arm extensions 1834 of platform 808 releasably secure sensor module 504 in position. Module 2200 is coupled with connector 2300, sharp module 2500 and sensor (not shown) such that during assembly they can be removed together as sensor module 504.

### Example Embodiment of Applicator Housing

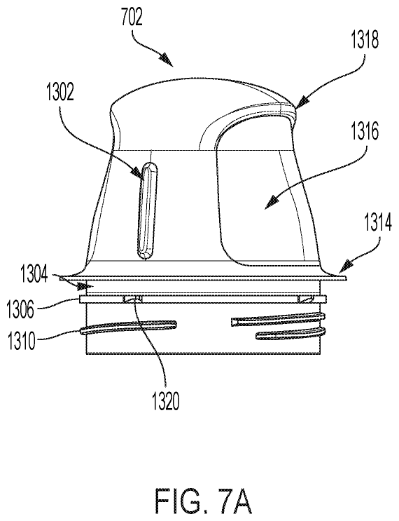

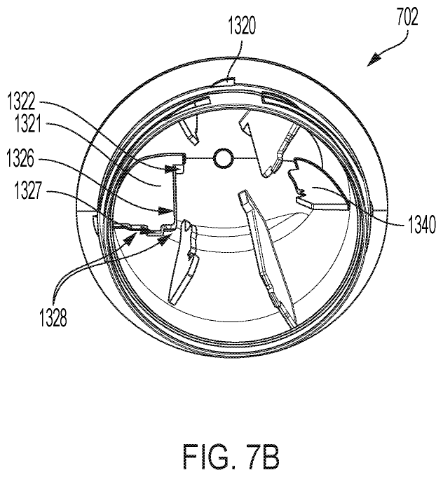

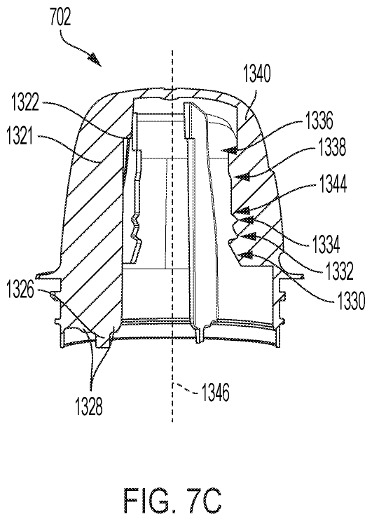

FIG. 7A is side view depicting an example embodiment of the applicator housing 702 that can include an internal cavity with support structures for applicator function. A user can push housing 702 in a distal direction to activate the applicator assembly process and then also to cause delivery of sensor control device 102, after which the cavity of housing 702 can act as a receptacle for a sharp. In the example embodiment, various features are shown including housing orienting feature 1302 for orienting the device during assembly and use. Tamper ring groove 1304 can be a recess located around an outer circumference of housing 702, distal to a tamper ring protector 1314 and proximal to a tamper ring retainer 1306. Tamper ring groove 1304 can retain a tamper ring so users can identify whether the device has been tampered with or otherwise used. Housing threads 1310 can secure housing 702 to complimentary threads on cap 708 (FIGS. 4A and 4B) by aligning with complimentary cap threads and rotating in a clockwise or counterclockwise direction. A side grip zone 1316 of housing 702 can provide an exterior surface location where a user can grip housing 702 in order to use it. Grip overhang 1318 is a slightly raised ridge with respect to side grip zone 1316 which can aid in ease of removal of housing 702 from cap 708. A shark tooth 1320 can be a raised section with a flat side located on a clockwise edge to shear off a tamper ring (not shown), and hold tamper ring in place after a user has unscrewed cap 708 and housing 702. In the example embodiment four shark teeth 1320 are used, although more or less can be used as desired.

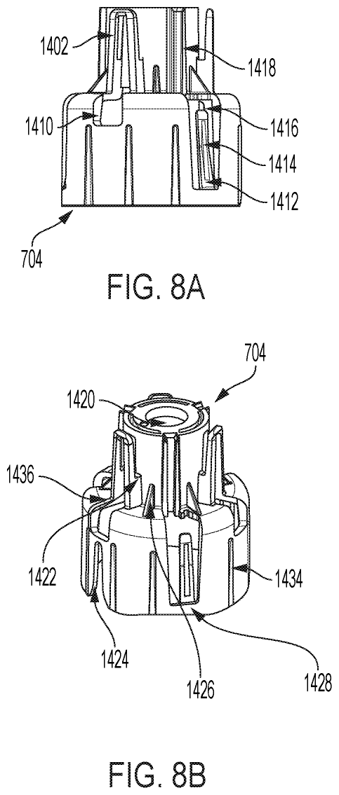

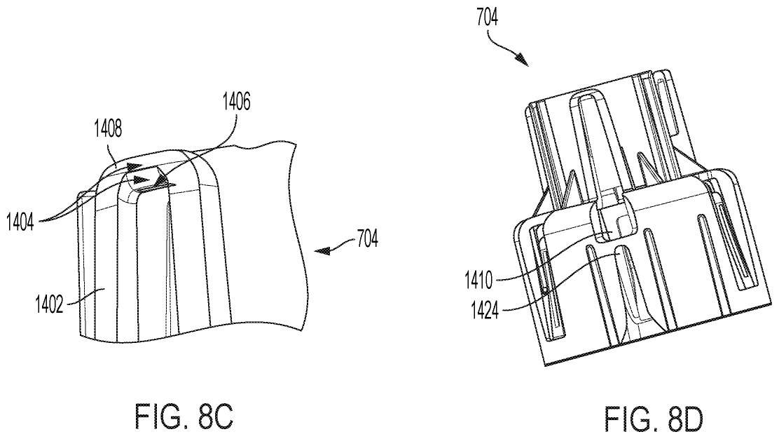

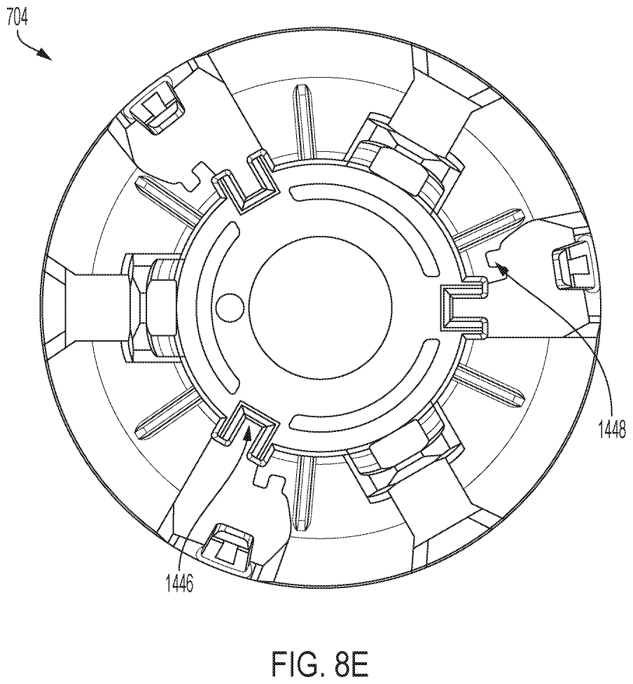

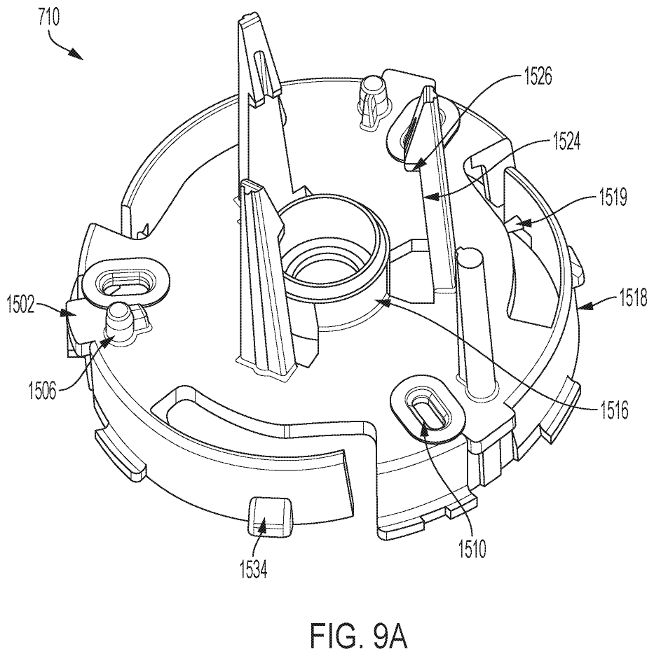

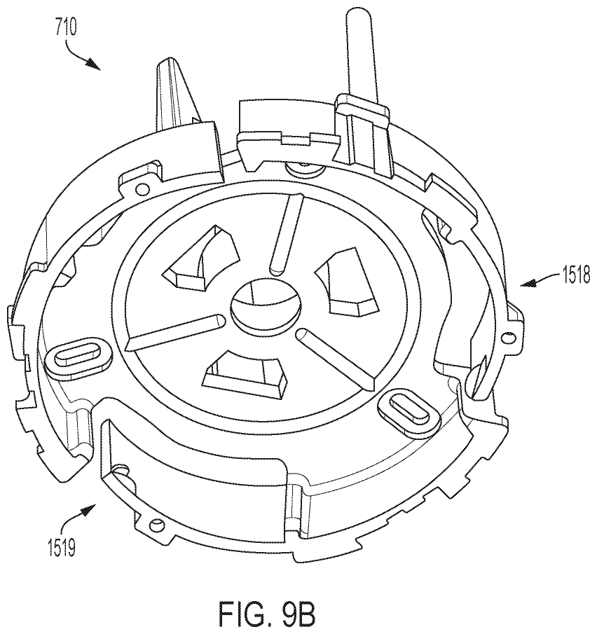

FIG. 7B is a perspective view depicting a distal end of housing 702. Here, three housing guide structures (or “guide ribs”) 1321 are located at 120 degree angles with respect to each other, and at 60 degree angles with respect to locking structures (or “locking ribs”) 1340, of which there are also three at 120 degree angles with respect to each other. Other angular orientations, either symmetric or asymmetric, can be used, as well as any number of one or more structures 1321 and 1340. Here, each structure 1321 and 1340 is configured as a planar rib, although other shapes can be used. Each guide rib 1321 includes a guide edge (also called a “sheath guide rail”) 1326 that can pass along a surface of sheath 704 (e.g., guide rail 1418 described with respect to FIG. 8A). An insertion hard stop 1322 can be a flat, distally facing surface of housing guide rib 1321 located near a proximal end of housing guide rib 1321. Insertion hard stop 1322 provides a surface for a sensor electronics carrier travel limiter face 1420 of a sheath 704 (FIG. 8B) to abut during use, preventing sensor electronics carrier travel limiter face 1420 from moving any further in a proximal direction. A carrier interface post 1327 passes through an aperture 1510 (FIG. 9A) of device carrier 710 during an assembly. A device carrier interface 1328 can be a rounded, distally facing surface of housing guide ribs 1321 which interfaces with device carrier 710.

FIG. 7C is a side cross-section depicting an example embodiment of a housing. In the example embodiment, side cross-sectional profiles of housing guide rib 1321 and locking rib 1340 are shown. Locking rib 1340 includes sheath snap lead-in feature 1330 near a distal end of locking rib 1340 which flares outward from central axis 1346 of housing 702 distally. Each sheath snap lead-in feature 1330 causes detent snap round 1404 of detent snap 1402 of sheath 704 as shown in FIG. 8C to bend inward toward central axis 1346 as sheath 704 moves towards the proximal end of housing 702. Once past a distal point of sheath snap lead-in feature 1330, detent snap 1402 of sheath 704 is locked into place in locked groove 1332. As such, detent snap 1402 cannot be easily moved in a distal direction due to a surface with a near perpendicular plane to central axis 1346, shown as detent snap flat 1406 in FIG. 8C.

As housing 702 moves further in a proximal direction toward the skin surface, and as sheath 704 advances toward the distal end of housing 702, detent snaps 1402 shift into the unlocked grooves 1334, and applicator 150 is in an “armed” position, ready for use. When the user further applies force to the proximal end of housing 702, while sheath 704 is pressed against the skin, detent snap 1402 passes over firing detent 1344. This begins a firing sequence due to release of stored energy in the deflected detent snaps 1402, which travel in a proximal direction relative to the skin surface, toward sheath stopping ramp 1338 which is slightly flared outward with respect to central axis 1346 and slows sheath 704 movement during the firing sequence. The next groove encountered by detent snap 1402 after unlocked groove 1334 is final lockout groove 1336 which detent snap 1402 enters at the end of the stroke or pushing sequence performed by the user. Final lockout recess 1336 can be a proximally-facing surface that is perpendicular to central axis 1346 which, after detent snap 1402 passes, engages a detent snap flat 1406 and prevents reuse of the device by securely holding sheath 704 in place with respect to housing 702. Insertion hard stop 1322 of housing guide rib 1321 prevents sheath 704 from advancing proximally with respect to housing 702 by engaging sensor electronics carrier travel limiter face 1420.

### Example Embodiment of Applicator Sheath

FIGS. 8A and 8B are a side view and perspective view, respectively, depicting an example embodiment of sheath 704. In this example embodiment, sheath 704 can stage sensor control device 102 above a user's skin surface prior to application. Sheath 704 can also contain features that help retain a sharp in a position for proper application of a sensor, determine the force required for sensor application, and guide sheath 704 relative to housing 702 during application. Detent snaps 1402 are near a proximal end of sheath 704, described further with respect to FIG. 8C below. Sheath 704 can have a generally cylindrical cross section with a first radius in a proximal section (closer to top of figure) that is shorter than a second radius in a distal section (closer to bottom of figure). Also shown are a plurality of detent clearances 1410, three in the example embodiment. Sheath 704 can include one or more detent clearances 1410, each of which can be a cutout with room for sheath snap lead-in feature 1330 to pass distally into until a distal surface of locking rib 1340 contacts a proximal surface of detent clearance 1410.

Guide rails 1418 are disposed between sensor electronics carrier traveler limiter face 1420 at a proximal end of sheath 704 and a cutout around lock arms 1412. Each guide rail 1418 can be a channel between two ridges where the guide edge 1326 of housing guide rib 1321 can slide distally with respect to sheath 704.

Lock arms 1412 are disposed near a distal end of sheath 704 and can include an attached distal end and a free proximal end, which can include lock arm interface 1416. Lock arms 1412 can lock device carrier 710 to sheath 704 when lock arm interface 1416 of lock arms 1412 engage lock interface 1502 of device carrier 710. Lock arm strengthening ribs 1414 can be disposed near a central location of each lock arm 1412 and can act as a strengthening point for an otherwise weak point of each lock arm 1412 to prevent lock arm 1412 from bending excessively or breaking.

Detent snap stiffening features 1422 can be located along the distal section of detent snaps 1402 and can provide reinforcement to detent snaps 1402. Alignment notch 1424 can be a cutout near the distal end of sheath 704, which provides an opening for user alignment with sheath orientation feature of platform 808. Stiffening ribs 1426 can include buttresses, that are triangularly shaped here, which provide support for detent base 1436. Housing guide rail clearance 1428 can be a cutout for a distal surface of housing guide rib 1321 to slide during use.

FIG. 8C is a close-up perspective view depicting an example embodiment of detent snap 1402 of sheath 704. Detent snap 1402 can include a detent snap bridge 1408 located near or at its proximal end. Detent snap 1402 can also include a detent snap flat 1406 on a distal side of detent snap bridge 1408. An outer surface of detent snap bridge 1408 can include detent snap rounds 1404 which are rounded surfaces that allow for easier movement of detent snap bridge 1408 across interior surfaces of housing 702 such as, for example, locking rib 1340.

FIG. 8D is a side view depicting an example embodiment of sheath 704. Here, alignment notch 1424 can be relatively close to detent clearance 1410. Detent clearance 1410 is in a relatively proximal location on distal portion of sheath 704.

FIG. 8E is an end view depicting an example embodiment of a proximal end of sheath 704. Here, a back wall for guide rails 1446 can provide a channel to slidably couple with housing guide rib 1321 of housing 702. Sheath rotation limiter 1448 can be notches which reduce or prevent rotation of the sheath 704. In a general sense, the embodiments described herein operate by flattening and stretching a skin surface at a predetermined site for sensor insertion. Moreover, the embodiments described herein may also be utilized for other medical applications, such as, e.g., transdermal drug delivery, needle injection, wound closure stitches, device implantation, the application of an adhesive surface to the skin, and other like applications.

By way of background, those of skill the art will appreciate that skin is a highly anisotropic tissue from a biomechanical standpoint and varies largely between individuals. This can affect the degree to which communication between the underlying tissue and the surrounding environment can be performed, e.g., with respect to drug diffusion rates, the ability to penetrate skin with a sharp, or sensor insertion into the body at a sharp-guided insertion site.

### Example Embodiments of Device Carriers

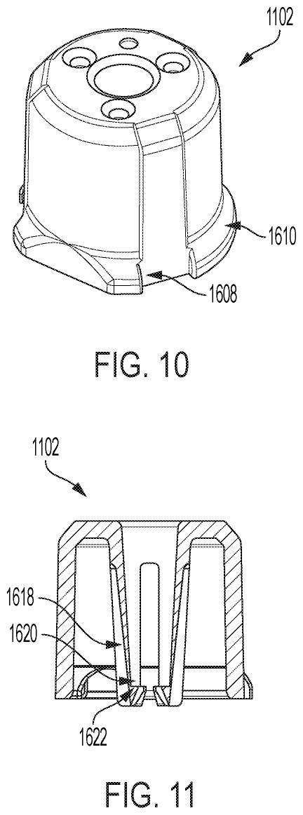

FIG. 9A is a proximal perspective view depicting an example embodiment of device carrier 710 that can retain sensor electronics within applicator 150. It can also retain sharp carrier 1102 with sharp module 2500. In this example embodiment, carrier 710 generally has a hollow round flat cylindrical shape, and can include one or more deflectable sharp carrier lock arms 1524 (e.g., three) extending proximally from a proximal surface surrounding a centrally located spring alignment ridge 1516 for maintaining alignment of spring 1104. Each lock arm 1524 has a detent or retention feature 1526 located at or near its proximal end. Shock lock 1534 can be a tab located on an outer circumference of device carrier 710 extending outward and can lock device carrier 710 for added safety prior to firing. Rotation limiter 1506 can be a proximally extending relatively short protrusion on a proximal surface of device carrier 710 which limits rotation of carrier 710. Sharp carrier lock arms 1524 can interface with sharp carrier 1102 as described with reference to FIGS. 10 and 11 below.

FIG. 9B is a distal perspective view of device carrier 710. Here, one or more sensor electronics retention spring arms 1518 (e.g., three) are normally biased towards the position shown and include a detent 1519 that can pass over the distal surface of electronics housing 706 of device 102 when housed within recess or cavity 1521. In certain embodiments, after sensor control device 102 has been adhered to the skin with applicator 150, the user pulls applicator 150 in a proximal direction, i.e., away from the skin. The adhesive force retains sensor control device 102 on the skin and overcomes the lateral force applied by spring arms 1518. As a result, spring arms 1518 deflect radially outwardly and disengage detents 1519 from sensor control device 102 thereby releasing sensor control device 102 from applicator 150.

### Example Embodiments of Sharp Carriers

FIGS. 10 and 11 are a proximal perspective view and a side cross-sectional view, respectively, depicting an example embodiment of sharp carrier 1102. Sharp carrier 1102 can grasp and retain sharp module 2500 within applicator 150. Near a distal end of sharp carrier 1102 can be anti-rotation slots 1608 which prevent sharp carrier 1102 from rotating when located within a central area of sharp carrier lock arms 1524 (as shown in FIG. 9A). Anti-rotation slots 1608 can be located between sections of sharp carrier base chamfer 1610, which can ensure full retraction of sharp carrier 1102 through sheath 704 upon retraction of sharp carrier 1102 at the end of the deployment procedure.

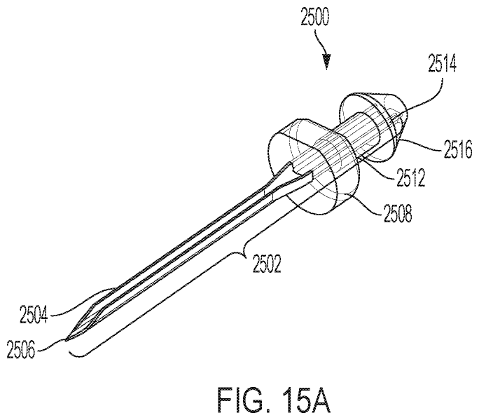

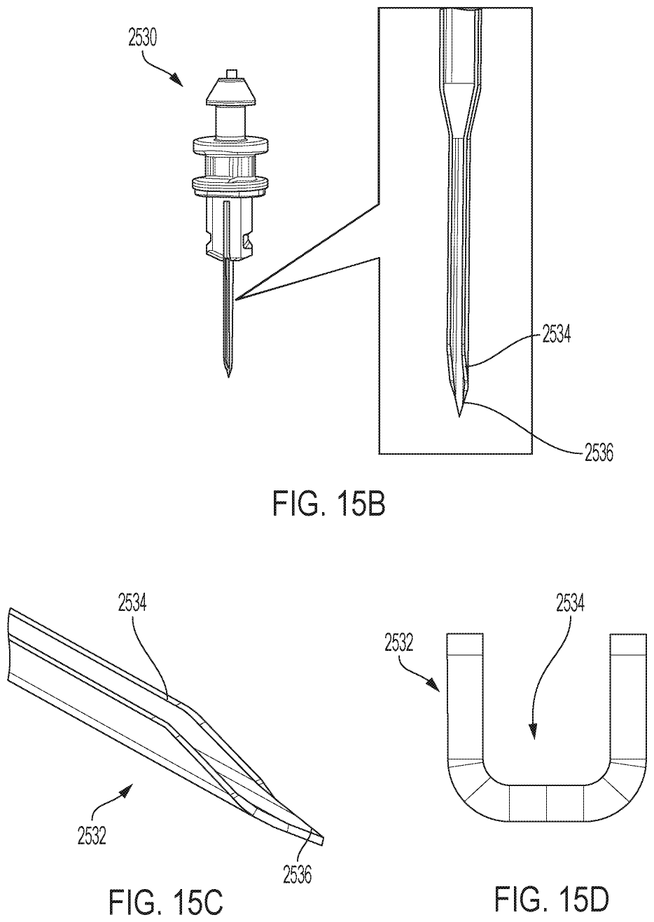

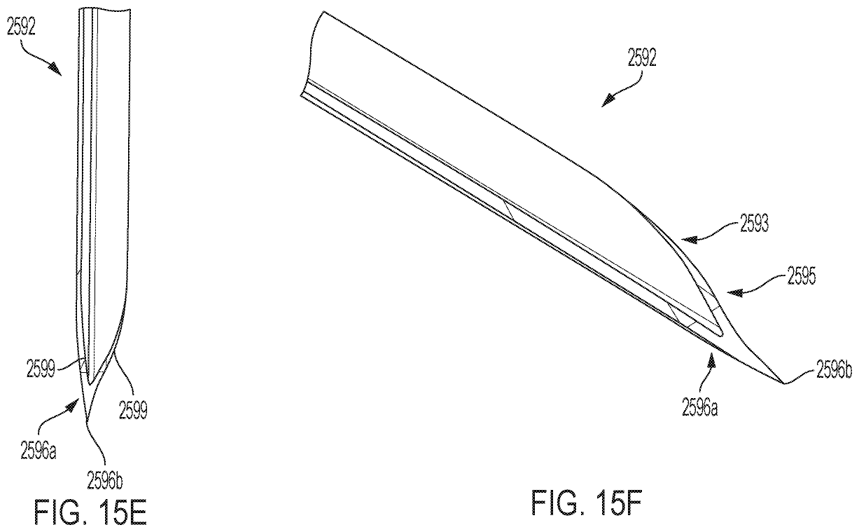

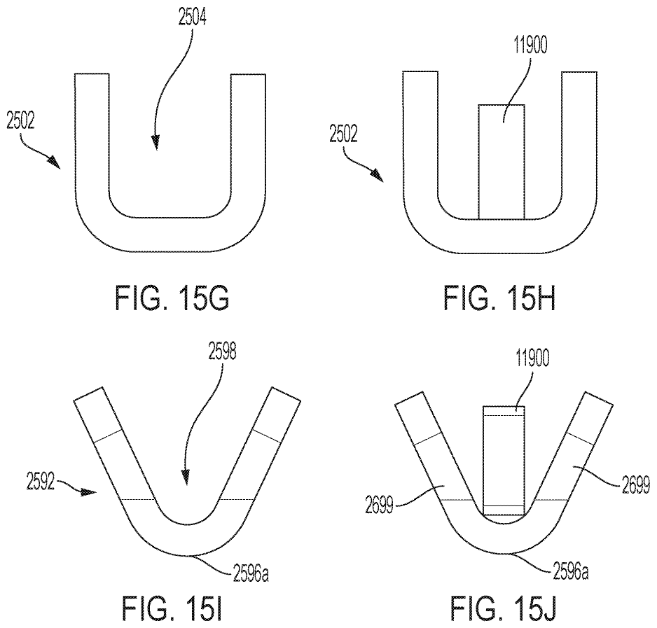

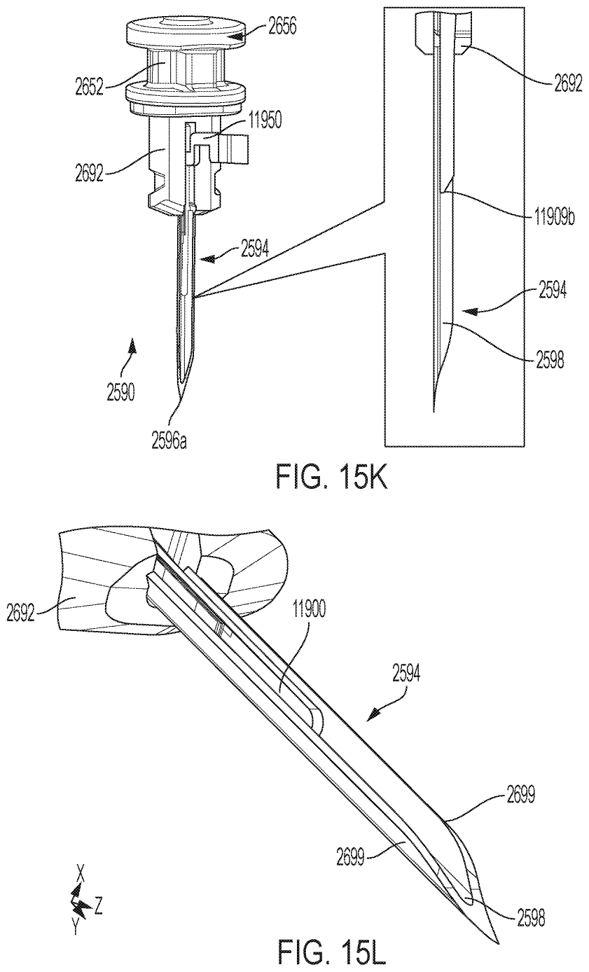

As shown in FIG. 11, sharp retention arms 1618 can be located in an interior of sharp carrier 1102 about a central axis and can include a sharp retention clip 1620 at a distal end of each arm 1618. Sharp retention clip 1620 can have a proximal surface which can be nearly perpendicular to the central axis and can abut a distally facing surface of sharp hub 2516 (FIG. 15A).

### Exemplary Sensor Modules

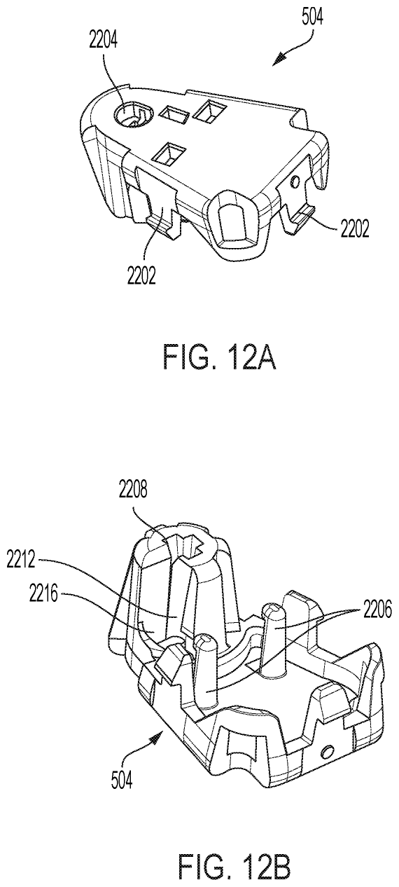

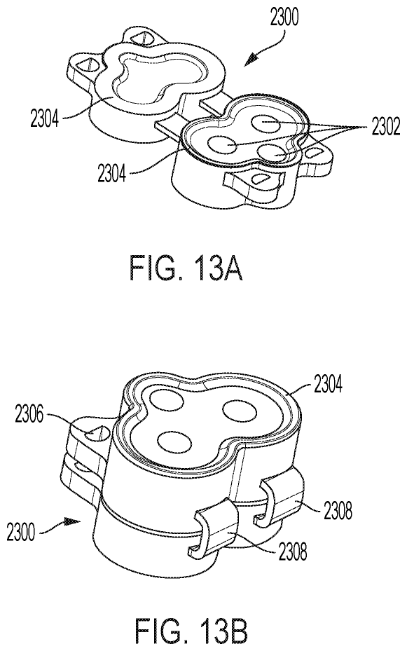

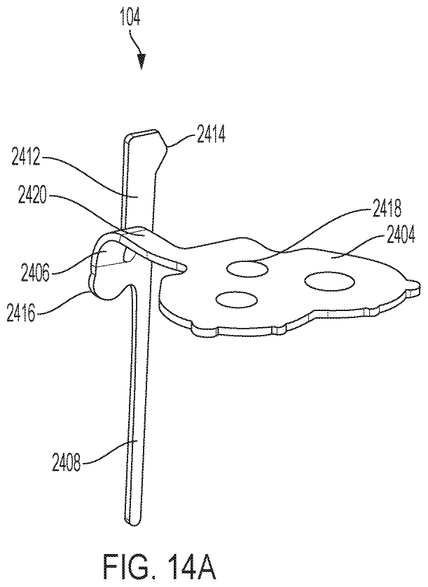

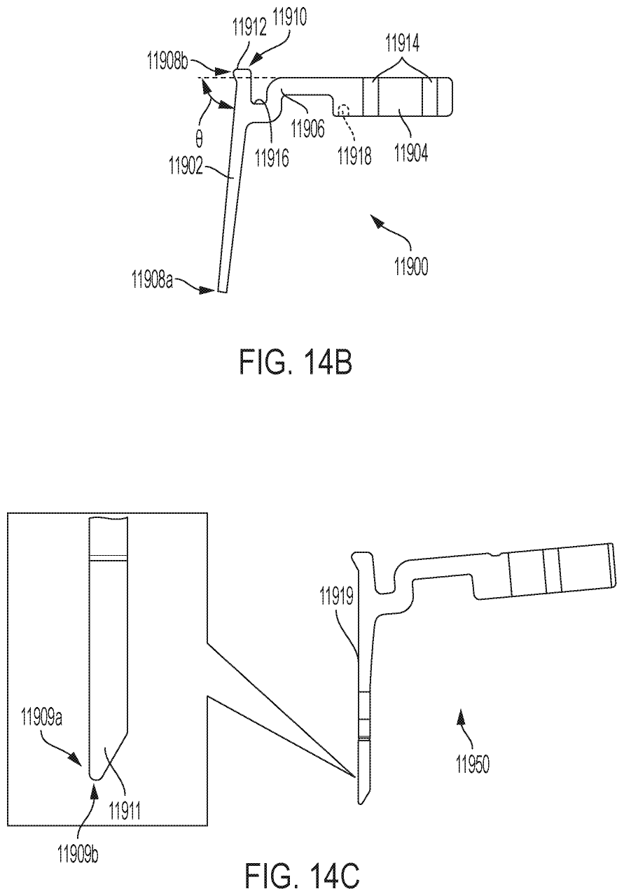

FIGS. 12A and 12B are a top perspective view and a bottom perspective view, respectively, depicting an example embodiment of sensor module 504. Module 504 can hold a connector 2300 (FIGS. 13A and 13B) and a sensor 104 (FIG. 14A). Module 504 is capable of being securely coupled with electronics housing 706. One or more deflectable arms or module snaps 2202 can snap into the corresponding features 2010 of housing 706. A sharp slot 2208 can provide a location for sharp tip 2502 to pass through and sharp shaft 2504 to temporarily reside. A sensor ledge 2212 can define a sensor position in a horizontal plane, prevent a sensor from lifting connector 2300 off of posts and maintain sensor 104 parallel to a plane of connector seals. It can also define sensor bend geometry and minimum bend radius. It can limit sensor travel in a vertical direction and prevent a tower from protruding above an electronics housing surface and define a sensor tail length below a patch surface. A sensor wall 2216 can constrain a sensor and define a sensor bend geometry and minimum bend radius.

FIGS. 13A and 13B are perspective views depicting an example embodiment of connector 2300 in an open state and a closed state, respectively. Connector 2300 can be made of silicone rubber that encapsulates compliant carbon impregnated polymer modules that serve as electrical conductive contacts 2302 between sensor 104 and electrical circuitry contacts for the electronics within housing 706. The connector can also serve as a moisture barrier for sensor 104 when assembled in a compressed state after transfer from a container to an applicator and after application to a user's skin. A plurality of seal surfaces 2304 can provide a watertight seal for electrical contacts and sensor contacts. One or more hinges 2308 can connect two distal and proximal portions of connector 2300.

FIG. 14A is a perspective view depicting an example embodiment of sensor 104. A neck 2406 can be a zone which allows folding of the sensor, for example ninety degrees. A membrane on tail 2408 can cover an active analyte sensing element of the sensor 104. Tail 2408 can be the portion of sensor 104 that resides under a user's skin after insertion. A flag 2404 can contain contacts and a sealing surface. A biasing tower 2412 can be a tab that biases the tail 2408 into sharp slot 2208. A bias fulcrum 2414 can be an offshoot of biasing tower 2412 that contacts an inner surface of a needle to bias a tail into a slot. A bias adjuster 2416 can reduce a localized bending of a tail connection and prevent sensor trace damage. Contacts 2418 can electrically couple the active portion of the sensor to connector 2300. A service loop 2420 can translate an electrical path from a vertical direction ninety degrees and engage with sensor ledge 2212 (FIG. 12B).

FIG. 14B is a side view of an example sensor 11900, according to one or more embodiments of the disclosure. The sensor 11900 may be similar in some respects to any of the sensors described herein and, therefore, may be used in an analyte monitoring system to detect specific analyte concentrations. As illustrated, the sensor 11900 includes a tail 11902, a flag 11904, and a neck 11906 that interconnects the tail 11902 and the flag 11904. The tail 11902 includes an enzyme or other chemistry or biologic and, in some embodiments, a membrane may cover the chemistry. In use, the tail 11902 is transcutaneously received beneath a user's skin, and the chemistry included thereon helps facilitate analyte monitoring in the presence of bodily fluids.

The tail 11902 may be received within a hollow or recessed portion of a sharp (not shown) to at least partially circumscribe the tail 11902 of the sensor 11900. As illustrated, the tail 11902 may extend at an angle Q offset from horizontal. In some embodiments, the angle Q may be about 85°. Accordingly, in contrast to other sensor tails, the tail 11902 may not extend perpendicularly from the flag 11904, but instead at an angle offset from perpendicular. This may prove advantageous in helping maintain the tail 11902 within the recessed portion of the sharp.

The tail 11902 includes a first or bottom end 11908a and a second or top end 11908b opposite the bottom end 11908a. A tower 11910 may be provided at or near the top end 11908b and may extend vertically upward from the location where the neck 11906 interconnects the tail 11902 to the flag 11904. During operation, if the sharp moves laterally, the tower 11910 will help pivot the tail 11902 toward the sharp and otherwise stay within the recessed portion of the sharp. Moreover, in some embodiments, the tower 11910 may provide or otherwise define a protrusion 11912 that extends laterally therefrom. When the sensor 11900 is mated with the sharp and the tail 11902 extends within the recessed portion of the sharp, the protrusion 11912 may engage the inner surface of the recessed portion. In operation, the protrusion 11912 may help keep the tail 11902 within the recessed portion.

The flag 11904 may comprise a generally planar surface having one or more sensor contacts 11914 arranged thereon. The sensor contact(s) 11914 may be configured to align with a corresponding number of compliant carbon impregnated polymer modules encapsulated within a connector.

In some embodiments, as illustrated, the neck 11906 may provide or otherwise define a dip or bend 11916 extending between the flag 11904 and the tail 11902. The bend 11916 may prove advantageous in adding flexibility to the sensor 11900 and helping prevent bending of the neck 11906.

In some embodiments, a notch 11918 (shown in dashed lines) may optionally be defined in the flag near the neck 11906. The notch 11918 may add flexibility and tolerance to the sensor 11900 as the sensor 11900 is mounted to the mount. More specifically, the notch 11918 may help take up interference forces that may occur as the sensor 11900 is mounted within the mount.

FIG. 14C illustrates one embodiment of a sensor 11950 including a modified tail 11919. In some embodiments, the first or bottom end 11909a is chiseled or tapered so as to form a bottom end 11909a having one or more beveled edges. In some embodiments, the bottom end 11909a comprises a single beveled edge 11911. More specifically, the sensor tail 11919 comprises a bottom end 11909a which forms a tip portion 11909b having a sharpened, V-shaped vertex. This sensor design can be advantageous in that reduces the overall footprint of the sensor 11950, and minimizes the size of skin penetration and puncture wound as it dilates the tissue during sensor insertion. Consequently, the risk of ESA created by wound trauma can be mitigated by the sensor's 11950 bottom end 11909a having a V-shaped tip portion 11909b with a sharpened vertex.

Generally, the sensor can be understood as including a tail, a flag, and a neck aligned along a planar surface having a vertical axis and a horizontal axis. The spring-like structure can be created by various orientations of turns in the bend of the neck of a sensor. Between the tail and the flag, the neck can include at least two turns in relation to the vertical axis providing a spring-like structure. The at least two turns can provide, in relation to an axis of the planar surface shared by the tail, the flag, and the neck, overlapping layers of the structure of the neck, where the neck itself remains unbroken. These overlapping turns make up the spring-like structure. In some embodiments, the overlapping layers of the neck are vertically-oriented. In some embodiments, the overlapping layers of the neck are horizontally-oriented.

The turns of the neck can be created by folding the neck of the sensor from a larger neck structure, laser cutting the sensor from a sheet of the material comprising the sensor, printing the sensor having the configuration with turns, stamping the sensor from a sheet of material of which the sensor is composed, or other suitable manufacturing processes for providing precision bends in the neck.

### Example Embodiments of Sharp Modules

FIG. 15A is a perspective view depicting an example embodiment of sharp module 2500 prior to assembly within sensor module 504 (FIGS. 12A and 12B). Sharp 2502 can include a distal tip 2506 which can penetrate the skin while carrying sensor tail in a hollow or recess of sharp shaft 2504 to put the active surface of the sensor tail into contact with bodily fluid. A hub push cylinder 2508 can provide a surface for a sharp carrier to push during insertion. A hub small cylinder 2512 can provide a space for the extension of sharp hub contact faces 1622 (FIG. 11). A hub snap pawl locating cylinder 2514 can provide a distal-facing surface of hub snap pawl 2516 for sharp hub contact faces 1622 to abut. A hub snap pawl 2516 can include a conical surface that opens clip 1620 during installation of sharp module 2500. Further details regarding embodiments of sharp modules, sharps, their components, and variants thereof, are described in U.S. Patent Publication No. 2014/0171771, which is incorporated by reference herein in its entirety and for all purposes.

FIG. 15B depicts another example embodiment of a sharp module 2530, and a callout view of the distal portion thereof, depicting the hollow or recess of sharp shaft 2534 and its distal tip 2536. FIG. 15C further illustrates a close-up side perspective view of distal tip 2536 which can penetrate the skin while carrying sensor tail (not shown) in a hollow or recess of sharp shaft 2534 to put the active surface of the sensor tail into contact with bodily fluid. The U-shaped implement of sharp 2532 is further illustrated in the cross-sectional view shown in FIG. 15D.

Example embodiments of a sharp designed to reduce trauma during a sensor insertion and retraction process will now be described. FIG. 15E is a close-up perspective view of a sharp 2592. In FIG. 15E, the sharp distal tip 2596a includes double beveled edges or transitions 2599 that adjoin at a proximal portion of the distal tip 2596a to form a distal tip 2596a having a vertex 2596b. Specifically, the double beveled edges 2599 are concavely sloped so as to form the sharp distal tip 2596a. Referring to FIG. 15E once more, the distal portion is provided with a concavely angled distal tip 2596a. As shown in FIG. 15F, the angled distal tip 2596a may be provided with a first concavely angled tip portion 2593 and a second steep-angled tip portion 2595. More specifically, the first concavely angled tip portion 2593 slopes into the second steep-angled tip portion 2595. The exemplary configuration, which includes multiple edges and faces, provides a sharp point to reduce penetration force, trauma, and bleeding for the subject. The sharp 2592 has a substantially V-shaped profile in this embodiment.

FIGS. 15G and 15I are cross-sectional view of two embodiments of the sharps described herein. FIG. 15G is a cross-sectional view of a previous embodiment, illustrating a substantially U-shaped cross-sectional area of sharp 2502. FIG. 15I is a cross-sectional view of an embodiment depicting a V-shaped cross-sectional area of sharp 2592, having a vertex 2596a. In some embodiments, the vertex 2596a includes a bottom portion of the cross-sectional area with no sharp edges. Additionally, the bottom portion of the cross-sectional area, unlike some U-shaped embodiments, such as the sharp embodiment illustrated in FIG. 15G, is not flattened. FIGS. 15H and 15J are cross-sectional views of the embodiments depicted in FIGS. 15G and 15I, respectively, illustrating the sharp embodiments supporting a sensor, for example, sensor 11900 or sensor 11950, respectively.

15K is a perspective view depicting an example embodiment of a sharp module 2590 having one or more beveled edges and V-shaped geometry configured to create a smaller opening in the skin relative to other sharps (e.g., sharp 2502 depicted in FIG. 15A). Sharp module 2590 is shown here prior to assembly with sensor module 504 (FIGS. 12A and 12B), and can include components similar to those of the embodiment described with respect to FIG. 15A, including sharp 2592, sharp shaft 2594, sharp distal tip 2596a, hub push cylinder (not shown), hub small cylinder 2652, hub snap pawl 2656 and hub snap pawl locating cylinder (not shown). Similar to the previously described sharp module 2500, and as shown in the callout view also depicted in FIG. 15K, sharp module 2590 can include a sharp shaft 2594 coupled to a distal end of the hub portion 2692 at a proximal end, sensor channel 2598 configured to receive at least a portion of an analyte sensor, such as analyte sensor 11950, for example, and a distal tip 2596a configured to penetrate a skin surface during the sensor insertion process.

In FIG. 15L, a front perspective view of sharp shaft 2594 is depicted, and includes a sharp module 2590 comprising one or more sidewalls 2699 and a longitudinally-extending sensor channel 2598 that together form the sharp's V-shaped cross-sectional area comprising a vertex 2596b. Further, the sensor channel 2598 is configured to receive at least a portion of any analyte sensor which has been described herein, such as analyte sensor 1900 or 11950, for example. In some embodiments, such as the embodiment which is illustrated in FIG. 15L, sensor channel 2598 is configured to receive at least a portion of analyte sensor 11900. According to one aspect of the embodiments, one or all of the sharp portion 2592, the sharp shaft 2594, and/or the sharp distal tip 2596a of a sharp 2592 can comprise one or more concave beveled edges.

Referring to FIGS. 15K and 15L, according to one aspect of the embodiment, one or more sidewalls 2699 that form sensor channel 2598 are disposed along sharp shaft 2594 and are adjacent to the distal tip 2596a. Specifically, the one or more sidewalls 2699 are disposed along the sharp shaft 2594 such that the terminus of the one or more sidewalls 2699 is distal to the terminus of the sensor channel 2598. In some embodiments, for example, the terminus of the one or more sidewalls 2699 extend from the vertex 2596b (as depicted in FIG. 15E) of the sharp distal tip 2596a. The terminus of the one or more sidewalls 2699 adjoin at the proximal end of the distal tip 2596a so as to form the one or more beveled edges thereof. The one or more beveled edges are configured such that they concavely slope and define the vertex 2596b of the distal tip 2596a. In some embodiments, the vertex 2596b is centrally located at the sharp distal tip 2596a. According to one aspect of the embodiment, a sharp 2592 can be characterized as having a sharpened V-shaped distal tip 2596a with all other edges comprising double beveled edges.

The V-shaped tip portion 2596a of the sharp 2592 is designed such that it provides less surface area and includes a reduced cross-sectional footprint relative to, for example, distal tip 2506 of sharp module 2500. The cross-sectional area of the distal tip 2596a is the smallest cross-sectional area of the sharp module 2590. During insertion, as the sharp 2592 moves into the skin surface, the sharp point geometry and V-shaped cross-sectional area of the sharp 2592, as illustrated in FIGS. 15E-15L, penetrate the skin by making a smaller wound due to the smaller size of the tip portion. With respect to sensor insertion, puncture wounds can contribute to ESA in sensors. In this regard, the embodiments described herein provide for less trauma and, consequently, a reduced risk of ESA result during the sensor insertion process.

Furthermore, it will be understood by those of skill in the art that the sharp 2592 embodiment described herein can similarly be used with any of the sensors described herein, including in vivo analyte sensors that are configured to measure an analyte level in a bodily fluid of a subject. Moreover, the sharp embodiments described herein can similarly be used with in vivo analyte sensors comprising a V-shaped tip. For example, in some embodiments, as shown in FIG. 15K, sharp 2592 can include a sensor channel 2598 configured to receive at least a portion of an analyte sensor 11950 with a V-shaped tip 11909b. Further, the V-shaped tip portion 11909b of the sensor 11950 is adjacent to the sharp 2592 and permits the sharp 2592 to create an insertion path for the sensor 11950. In this embodiment, the sharp design itself, and the positioning of the needle with respect to the sensor can be implemented such that the assembly causes less trauma during the insertion process. The distal section of the sensor body has a width sized to fit within the sensor channel 2598. For example, the V-shaped tip portion 11909b of the sensor 11950 is designed to have a complementary shape to the V-shaped cross-sectional area of the sharp 2592. In this embodiment, the V-shaped tip portion 11909b of the sensor 11950 is configured such that its tip portion 11909b is co-axial to the vertex 2596b of the sharp 2592 so as to better follow the distal tip 2596a as it penetrates the subject's skin.

### Example Embodiments of Sharpless Applicators

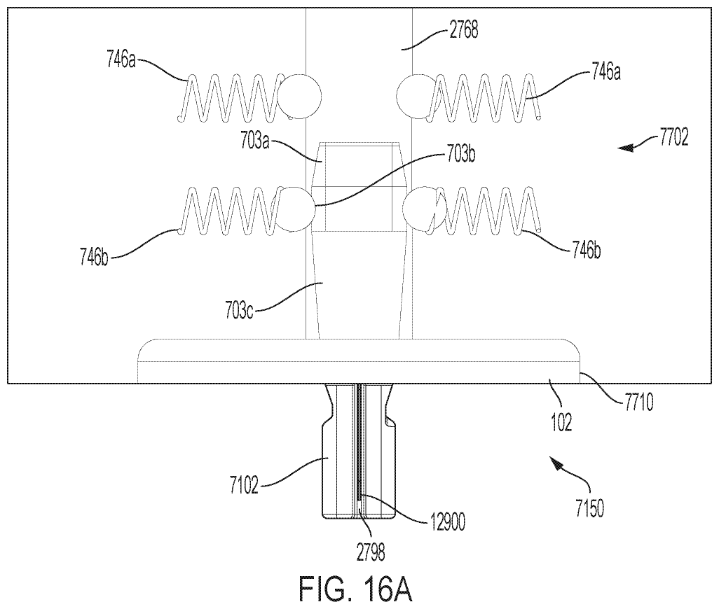

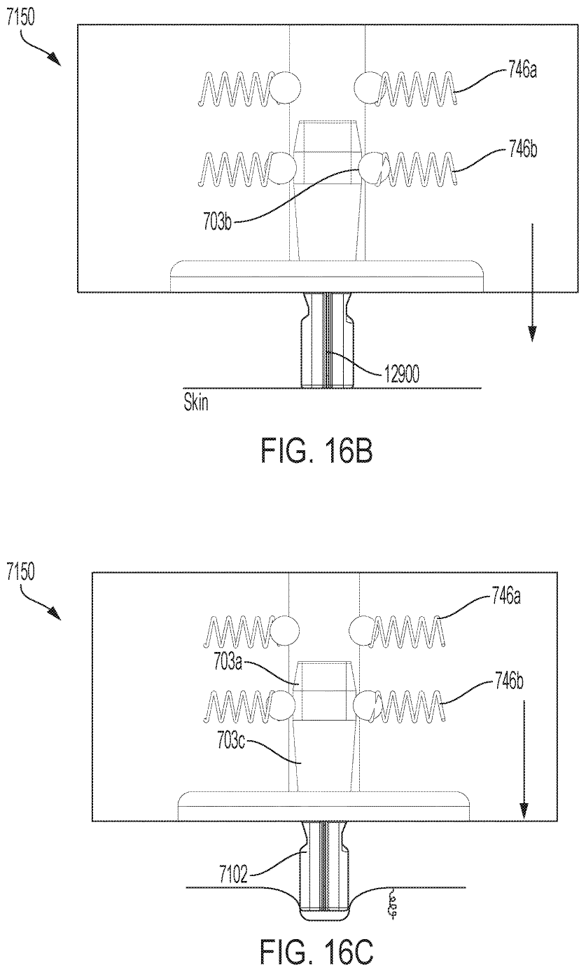

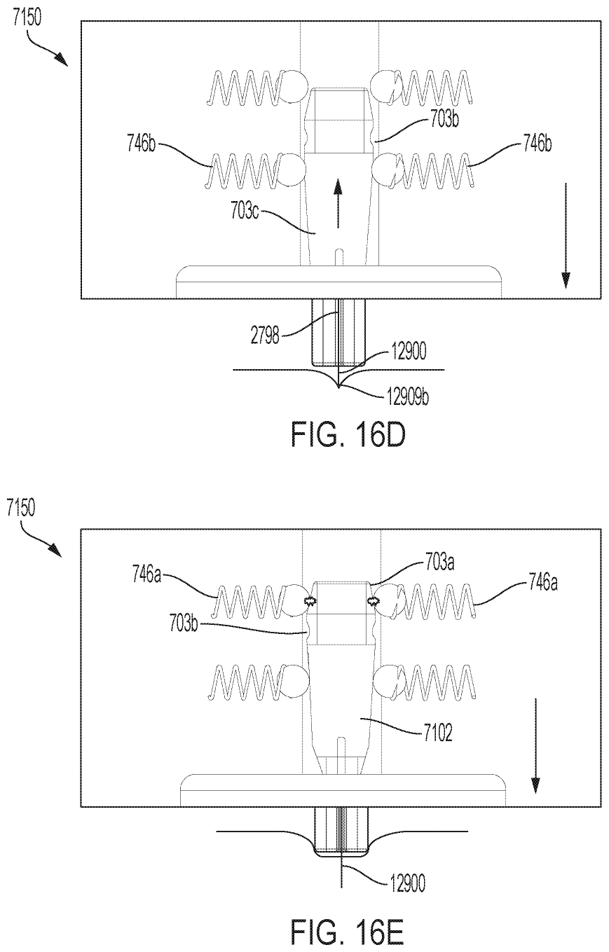

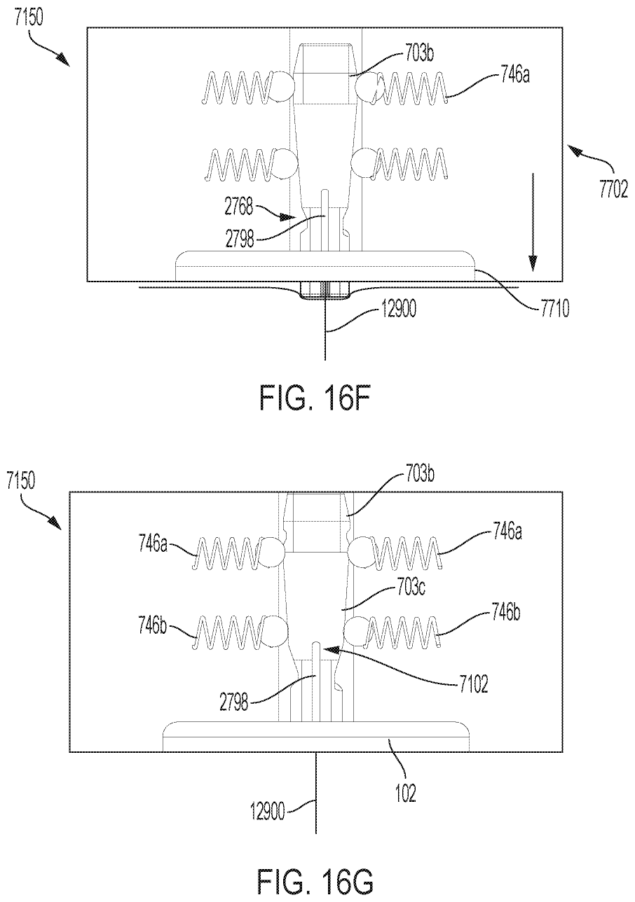

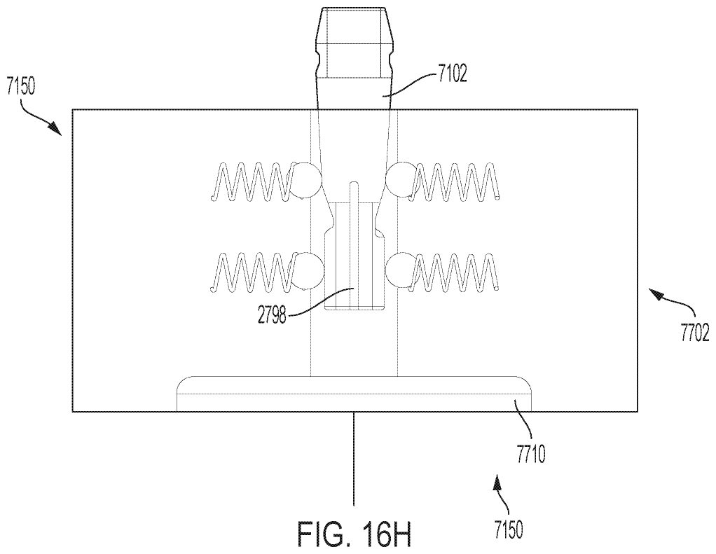

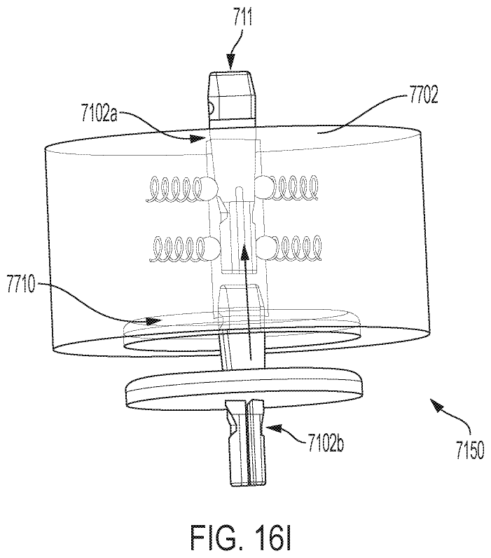

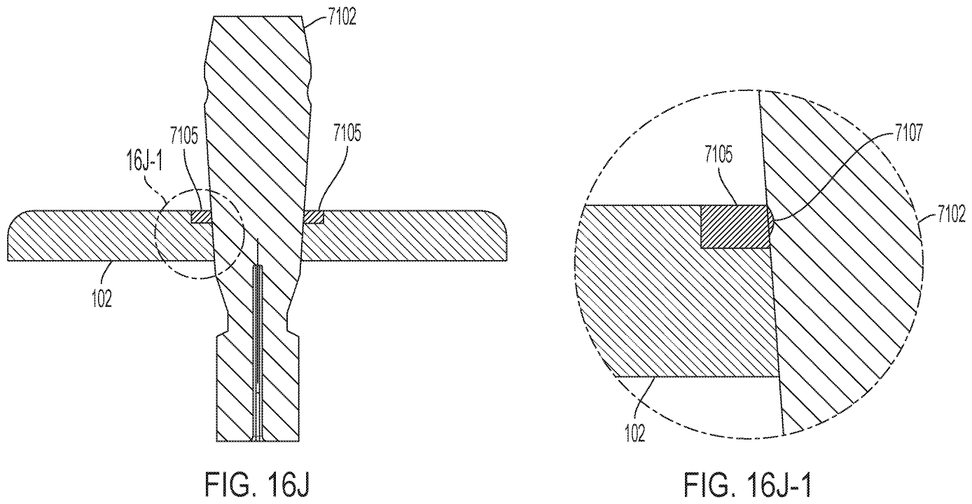

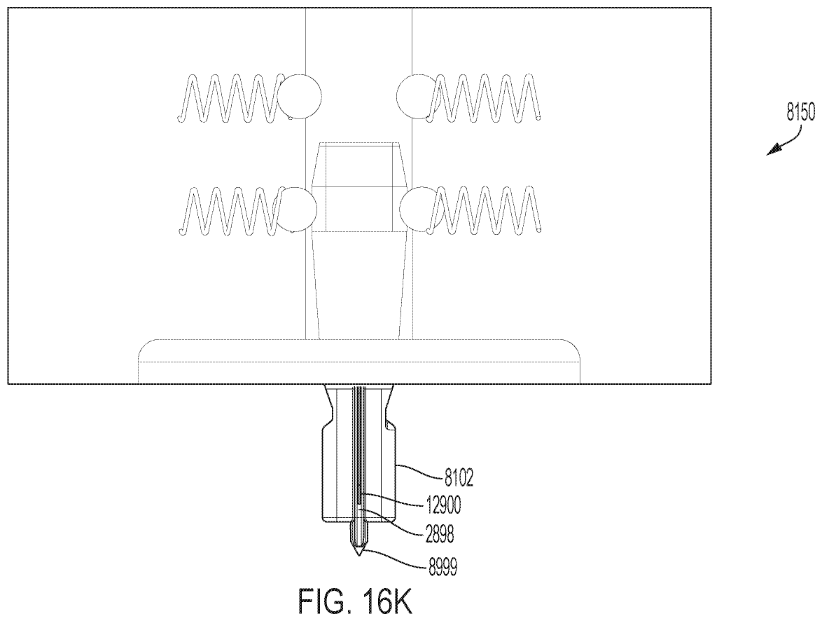

Example embodiments of various sharpless applicators will now be described. Referring first to FIG. 16A, a cross-sectional view of an example embodiment of a sharpless applicator 7150 (in an initial state) is depicted. According to one aspect of some embodiments, sharpless applicator assembly 7150 can include one or more of the following components: housing 7702, which can be movable between a proximal position and a distal position relative to a subject's skin; guide 7102; analyte sensor 12900; device carrier feature 7710 configured to releasably retain sensor control device 102; and a spring loaded system comprising a plurality of spring elements (e.g., spring elements 746a, 746b) which can be configured to engage with a proximal portion of guide 7102. Furthermore, in many embodiments, guide 7102 can be hollow and have a substantially cylindrical shape. In many embodiments, guide 7102 can also include a plurality of ramp surfaces 703a, 703c (e.g., two) with a groove or detent section 703b disposed in between the ramp surfaces.

According to another aspect of some embodiments, device carrier feature 7710 can have a concave or partially concave geometry that is complementary to the shape of sensor control device 102. For example, in some embodiments, device carrier feature 7710 can comprise a hollow round flat cylindrical shape configured to releasably contain sensor control device 102.

Referring still to FIG. 16A, in some embodiments, guide 7102 can comprise a sensor support channel 2798, wherein at least a portion of the sensor support channel 2798 can be disposed in a distal end of guide 7102. In some embodiments, the distal end of guide 7102 can be configured for placement on the skin of the subject. According to some embodiments, a first end of sensor support channel 2798 can terminate at the distal end of guide 7102. In some embodiments, the proximal end of guide 7102 can be hollow. In other embodiments, the proximal end of the guide 7102 can be solid.

According to another aspect of some embodiments, a width of at least a portion of the proximal end of the guide 7102 can be greater than a width of the distal end of guide 7102. Further, in some embodiments, a first ramp 703a can be located at the proximal end of guide 7102, whereas the second ramp 703c can be located distally relative to the first ramp 703a and groove section 703b. According to some embodiments, a width of the groove or detent section 703b of the guide 7102 can be less than the width of the first ramp 703a and the second ramp 703c (at least at the portion which connects the sections).

Referring still to FIG. 16A, according to another aspect of some embodiments, an analyte sensor 12900 can be at least partially disposed within sensor support channel 2798, and supported by guide 7102. Analyte sensor 12900 can include components similar to those of the embodiment described with respect to FIG. 14C. In many embodiments, analyte sensor 12900 comprises a tip portion 12909b (as depicted in FIG. 16D) with sufficient sharpness to initiate and complete insertion without the need for a separate sharp. Accordingly, analyte sensor 12900 can include a predetermined free length so as to provide the necessary sensor stiffness to facilitate effective insertion into the subject's skin. According to one aspect of the embodiments, stiffness of the sensor can be determined by free length of the sensor (e.g., an analyte sensor with a shorter free length will be stiffer). This can mitigate certain effects from “skin tenting,” a phenomenon that occurs when a sharp tip contacts skin and, prior to penetration, the skin deforms inwardly into the body. As a result of “skin tenting,” if the sharp or sensor is not sufficiently stiff, the sharp or sharpened sensor may fail to create a sufficiently large insertion point or misposition the sensor due to deflection.

Referring back to FIG. 16A, according to some embodiments, sharpless applicator 7150 can include an alignment shaft 2768 for maintaining the longitudinal alignment of the guide 7102 such that guide 7102 moves axially within the alignment shaft 2768 while it interfaces with a spring loaded system. In some embodiments, alignment shaft 2768 has a cylindrical geometry. According to one aspect of some embodiments, guide 7102 can be partially disposed within alignment shaft 2768 of the device support 7710 when the sharpless applicator 7150 is in the initial stage. According to another aspect of some embodiments, guide 7102 can be entirely disposed within alignment shaft 2768 after the sharpless applicator 7150 has completed the insertion and retraction steps.

According to another aspect of some embodiments, a plurality of spring elements can be disposed in or along an interior portion of alignment shaft 2768 to facilitate movement or positioning of guide 7102. In particular, the plurality of spring elements can be configured to control a position of guide 7102, increase or decrease the speed of guide 7102 during insertion and/or retraction, and also limit the free length of the sensor during various stages of operation. In this regard, the sensor stiffness can be maintained to aid in effective insertion. In some embodiments, a plurality of low friction rollers, ball-and-plunger sets, or an equivalent thereof, can be utilized to provide spring elements for the spring-loaded system. As shown in FIG. 16A, two pairs of ball-plunger sets 746a, 746b are utilized, an upper ball-plunger set 746a and a bottom ball-plunger set 746b, wherein each set can comprise a first ball-plunger that interacts with a left side of guide 7102, and a second ball-plunger that interacts a right side of guide 7102. Specifically, each ball-plunger set 746a, 746b can comprise a spring with one end anchored along the alignment shaft 2768 (or, alternatively, another stationary structure of sharpless applicator 7150, such as housing 7702), wherein a second end of the spring includes a ball structure configured to engage with a portion or side of guide 7102 as it travels along alignment shaft 2768. According to another aspect of the embodiments, each spring can be in a partially or fully compressed and/or expanded state during various stages of operation of the sharpless applicator 7150. According to yet another aspect of the embodiments, each of the spring elements can be configured to compress and/or expand at different times. For example, in some embodiments, at a certain stage of retraction, upper ball-plunger set 746a can be positioned further inward toward the center of alignment shaft 2768 relative to the bottom ball-plunger set 746b. In this regard, the sharpless applicator assembly retraction mechanism relies at least in part on an upward force of the subject's skin during insertion, combined with the ball-plunger interaction with the ramped surfaces 703a, 703c of the guides 7102 to allow for retraction of the guide 7102 into a container 711 within the housing 7702. Those of skill in the art will also appreciate that the embodiments of the present disclosure can allow for re-usability of sharpless applicator 7150.

FIGS. 16B-16H are various cross-sectional views depicting an example embodiment of a sharpless applicator assembly 7150 during various stages of operation.

FIG. 16B is a cross-sectional view of an example embodiment of a sharpless applicator assembly 7150 for insertion of an analyte sensor 12900 in a subject. In the initial state, the distal end of the guide 7102 is in contact with the subject's skin surface. In this initial state, as shown in FIG. 16B, the bottom ball-plunger set 746b is engaged with the groove or detent section 703b of guide 7102. According to one aspect of the embodiments, the ball portion of each ball-plunger set of the bottom spring elements 746b is in contact with the detent section 703b, and the corresponding spring of each ball-plunger set is in a partially compressed state. As shown in FIG. 16B, a force is applied to sharpless applicator 7150 causing it to move in a distal direction towards the subject's skin.

FIG. 16C is a cross-sectional view showing sharpless applicator assembly 7150 as applicator 7150 continues moving distally (e.g., in a downward direction) and creates a force in the distal direction against the skin. Meanwhile, according to one aspect of the embodiments, the bottom ball-plunger set 746b exerts a force on groove section 703b of guide 7102, and prevents guide 7102 from moving until a sufficient force in the proximal direction is received. As applicator 7150 continues to move distally, distal end of guide 7102 continues to push down on the subject's skin. As a result, as can be seen in FIG. 16C, the skin surface deforms from the force of guide 7102, and subsequently exerts a counterforce in the proximal direction against guide 7102. Consequently, the bottom ball-plunger set 746b begins to disengage from the groove section 703b.

Turning to FIG. 16D, as applicator assembly 7150 continues to move in a distal direction, the downward force from applicator 7150 further deforms the skin, which in turn increases the skin's counterforce exerted in the proximal direction against guide 7102. The upward force exerted by the skin exceeds the force imparted from the spring loaded system to hold guide 7102 in place. Consequently, bottom ball-plunger set 746b disengages from groove section 703b of guide 7102, as guide 7102 begins to travel in a proximal direction inside alignment shaft. According to another aspect of some embodiments, ramped surface of guide 7102 and the counterforce exerted by the deformed or stretched skin result in acceleration of guide 7102 in the proximal (e.g., upward) direction, and subsequently expose the sharp tip portion 12909b of analyte sensor 12900 disposed within the sensor support channel 2798. The acceleration of the guide 7102 increases the velocity of the skin relative to the sensor 12900, as the skin follows guide 7102. In this regard, the sharpless applicator assembly 7150 utilizes the skin deformation, or skin tenting, during insertion as a source of potential energy to further load the guide 7102 and increase relative velocity between skin and sensor. This increase in velocity can aid in insertion effectiveness. As the sharp sensor 12900 protrudes from the sensor support channel 2798, it begins to insert itself into the skin.

FIG. 16E shows the applicator assembly 7150 as it continues moving in a distal direction towards the skin. At the stage illustrated in FIG. 16E, guide 7102 continues to accelerate in the proximal direction until the first ramp 703a engages the upper ball-plunger set 746a, which impedes the motion of guide 7102. This ensures that sensor length during insertion is small enough to maintain the desired stiffness for effective insertion and avoid sensor bending. At this stage, the contact between the guide 7102 and the upper ball-plunger set 746a increases the force applied to skin, thereby causing further skin deformation. As this occurs, the counterforce created by the skin overcomes the force from the ball-plunger sets 746a, 746b, causing guide 7102 to accelerate in the proximal direction (e.g., away from the skin).

FIG. 16F shows the applicator assembly 7150 as it continues moving downward after sensor insertion. Guide 7102 continues to travel in a proximal direction into housing 7702 due to the counterforce generated by the skin. According to some embodiments, at this stage, sensor insertion can continue with more of the sensor length entering the skin of the subject. Further, at this stage, the upper ball-plunger set 746a sits in the guide grooves 703b. In this stage, the guide 7102 is still partially protruding from its distal end and is not fully retracted into the applicator 7150.

FIG. 16G illustrates sharpless applicator assembly 7150 in a sensor fully-inserted stage. In some embodiments, the motion of housing is slowed or stopped by sensor control device 102 contacting the skin. According to one aspect of the embodiments, at the stage depicted in FIG. 16G, analyte sensor 12900 has reached the desired insertion depth in the subject's skin. In some embodiments, distal end of guide 7102 can be flush with bottom surface of sensor control device 102. As shown in FIG. 16G, guide groove section 703b has now been advanced to a position proximal to the upper ball-plunger set 746a. As such, both ball-plunger sets 746a, 746b are in contact with the second ramp 703c of guide 7102. This can impart a force on guide 7102 in a proximal direction, thereby pushing guide 7102 further into the housing 7702 and away from device carrier 7710. With respect to the guide ramp shapes and ball-plunger forces, those of skill in the art will appreciate that certain embodiments of the guide ramps and ball-plungers can be dimensioned and configured to provide optimization for increased axial force in either a distal or proximal direction to ensure insertion and/or retraction. For example, if certain guide ramp surfaces are more sloped, this can cause an increase in insertion and/or retraction speed. Alternatively, certain guide ramp shapes may increase the initial acceleration.

FIG. 16H is a cross-sectional view showing the sharpless applicator assembly 7150 in a retraction state. As depicted in FIG. 16H, sensor 12900 has been inserted into the subject's skin to the desired insertion depth, and guide 7102 has been retracted fully into the applicator 7150 and away from user access. The sharpless applicator assembly 7150 is further advantageous because it does not contain a sharp and is therefore not considered a biohazard.

In addition, as seen in FIG. 16I, according to some embodiments, sharpless applicator assembly 7150 can include a container 711 either coupled with or inside applicator 7150. According to some embodiments, container 711 can be configured to collect used guides. In some embodiments where sharpless applicator assembly 7150 is reusable, the user may reload a new guide and sensor control device into the distal end of the applicator. By doing this, a force in the proximal direction is exerted on the used guide 7102a, causing it to push upwards into the applicator 7150 until it is collected in the container 711. In some embodiments, the container 711 is designed and configured to collect a plurality of used guides. Alternatively, the used guide 7102a may be ejected from an aperture at the proximal surface of the applicator 7150 every time a user loads a new guide 7102b.

FIG. 16J depicts a guide-sensor control device assembly, which can be configured to be loaded into a reusable sharpless applicator, such as those depicted in FIGS. 16A to 16I. As can be seen in call-out FIG. 16J-1, to prevent contaminants from entering an interior of sensor control device 102, a seal 7105 can be disposed at an interface between guide 7102 and sensor control device 102. According to one aspect of the embodiments, seal 7105 can comprise an overmolded elastomeric material. In some embodiments, seal 7105 can comprise a single annular ring configured to interface with guide 7102. In other embodiments, seal 7105 can comprise a plurality of discrete elastomeric components configured to interface a corresponding plurality of locations along guide 7102. According to another aspect of some embodiments, seal 7105 can comprise at least one concave surface 7107 configured to interface with guide 7102. In some embodiments, sensor control device 102 can comprise a housing constructed from a hard plastic material different from the elastomeric material of seal 7105. In other embodiments, housing of sensor control device 102 can be constructed from the same material of seal 7105, such that the housing and seal have a unitary construction. Those of skill in the art will appreciate that guide 7102, or at least some portions thereof, can comprise an elastomeric material or have one or more concave surfaces to provide for a barrier against contaminants entering the interior of sensor control device 102.

Further, FIG. 16K depicts a sharpless applicator assembly 8150, which is similar to the sharpless applicator assembly 7150 illustrated in FIGS. 16A-16I, except that sharpless applicator assembly 8150 further comprises guide 8102 having a short sharp distal end 8999. Specifically, guide 8102 comprises sharp distal end 8999 with sufficient sharpness to initiate skin penetration and facilitate insertion of the sensor 12900 to its fully-inserted stage or desired insertion depth. In some embodiments, a first end of sensor support channel 2898 extends into and terminates at the sharp distal end 8999 of guide 8102.

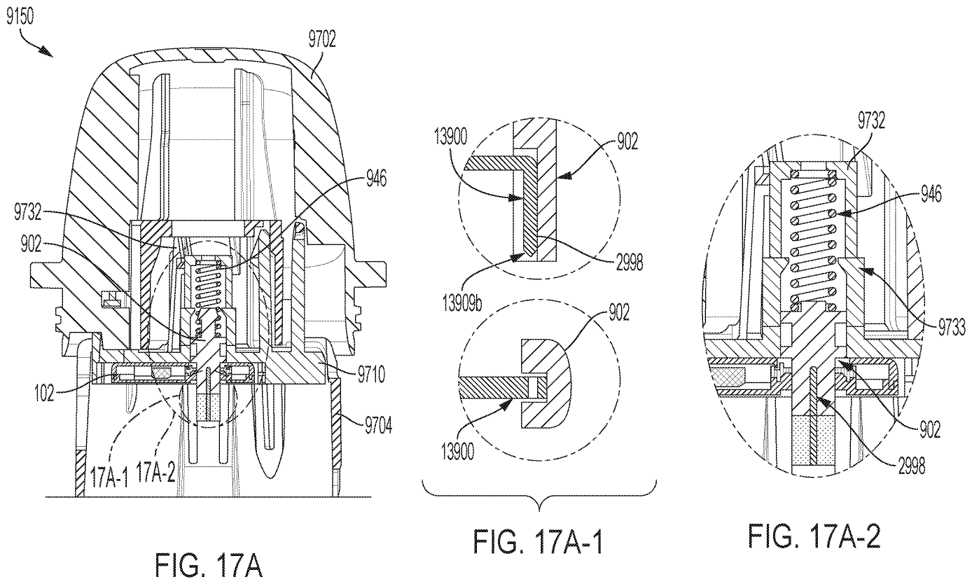

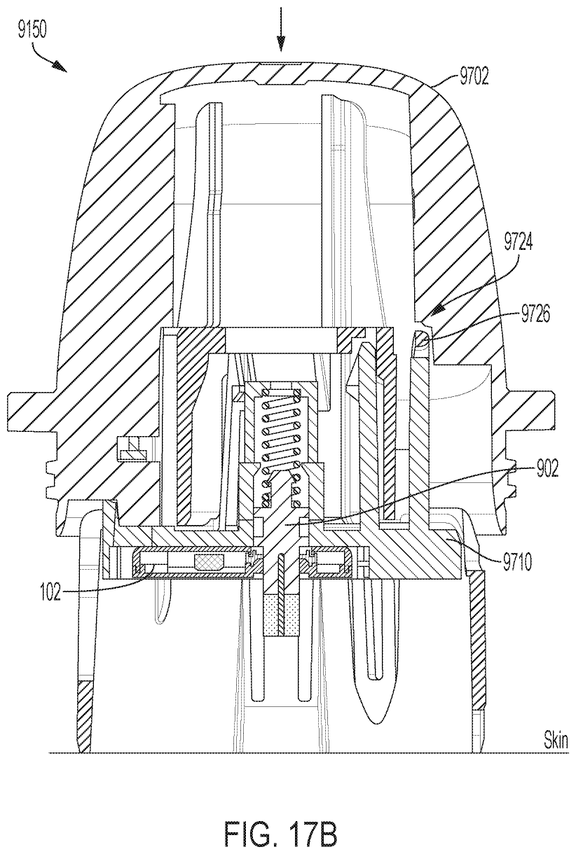

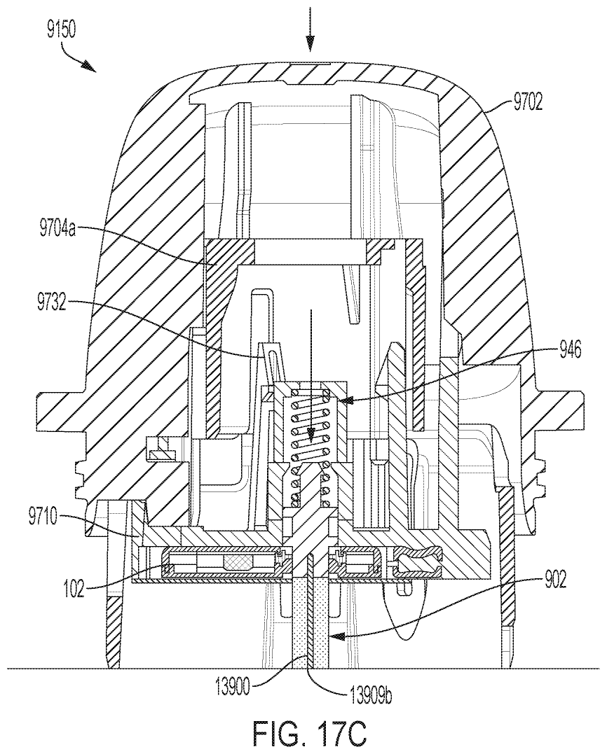

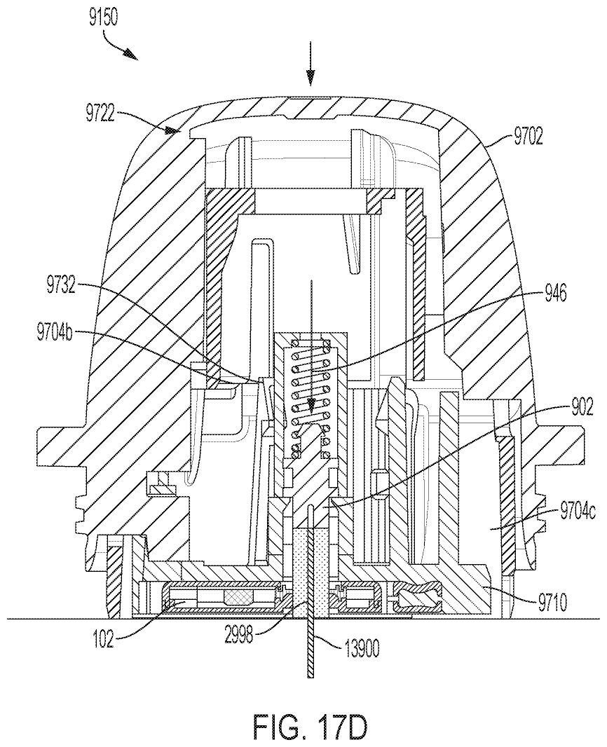

Another example embodiment of a sharpless applicator 9150 is illustrated in cross-section in FIG. 17A. Referring to FIG. 17A, various components of sharpless applicator 9150 will now be described. In particular, FIG. 17A depicts a cross-sectional view of a sharpless applicator 9150 in an initial state, shown along with two call-out views (FIG. 17A-1 and FIG. 17A-2), wherein sharpless applicator 9150 can comprise the following components: housing 9702, sheath 9704, guide 902, retraction spring 946, and device carrier 9710. FIG. 17A also depicts sensor control device 102 and sensor 13900 disposed entirely within sharpless applicator 9150. Those of skill in the art will understand that sharpless applicator 9150 can include any of the embodiments of housings, sheaths, device carriers, and/or analyte sensors described herein, or in other publications which have been incorporated by reference.

FIG. 17A illustrates sharpless applicator 9150 in an initial state, prior to insertion, in which housing 9702 is in a proximal position with respect to sheath 9704. According to an aspect of the embodiments, sheath 9704 is slidably coupled with and partially positioned within housing 9702. Although sheath 9704 and housing 9702 are shown in FIG. 17A as having a generally cylindrical geometry, those of skill in the art will appreciate that other geometries can be utilized. According to another aspect of the embodiments, as best seen in call-out FIG. 17A-2, spring 946 can comprise a distal end of the spring 946 in contact with a proximal end of guide 902, and a proximal end of spring 946 in contact with a spring retention element 9732. In some embodiments, spring retention element 9732 can be a proximally extending arm that protrudes from device carrier 9710, as described in further detail below. In other embodiments (not shown), spring retention element 9732 can comprise a feature of sheath 9704.

Referring still to FIG. 17A, according to an aspect of some embodiments, in the initial configuration, the retraction spring 946 may be in a semi-compressed state, i.e., not fully compressed, nor fully expanded, and pre-loaded against the guide 902 while housing 9702 is disposed proximally from sheath 9704. In other embodiments, retraction spring 946 may be fully compressed.

According to another aspect of some embodiments, sheath 9704 generally encloses or defines a cavity within which guide 902 and device carrier 9710 are moveable from a proximal position entirely within the applicator to a distal position. In many of the embodiments, device carrier 9710 is configured to releasably retain a sensor control device 102 having a distal surface for placement on the skin of the subject. In some embodiments, a guide locating ring (not shown) which receives one end of the guide 902 can be provided. Furthermore, in some embodiments, device carrier 9710 can comprise a proximally-extending latch 9733 (seen in call-out FIG. 17A-2) configured to retain guide 902 in a retracted position after insertion is complete. At least a portion of guide 902 extends through and is engaged with device carrier 9710 and sensor control device 102, and movable therein during insertion (e.g., before retraction is completed). As described above, in some embodiments, device carrier 9710 can also include one or more deflectable arms 9732 for retaining the retraction spring 946 in a compressed or partially compressed state. In some embodiments, the one or more deflectable arms 9732 may also engage with an exterior surface or wall of guide 902. The one or more movable arms 9732 may be maintained in engagement with the guide 9732 when the device carrier 9710 is in the proximal position.

According to another aspect of the embodiments, guide 902 can have a hollow and/or substantially cylindrical shape, and can include a sensor support channel or slot 2998, at least a portion of which is located in a distal portion of guide 902. In some embodiments, sensor support channel or slot 2998 can include a distal end that does not extend beyond the distal portion of guide 902. The proximal portion of guide 902 can be hollow and include a conical or partially conical surface.

According to another aspect of some embodiments, analyte sensor 13900 is at least partially disposed within sensor support channel or slot 2998 and, as best seen in call-out FIG. 17A-1, supported by one or more supporting walls of guide 902. The sensor tail of the analyte sensor 13900 may be disposed in the sensor support channel 2998. Analyte sensor 13900 can comprises a tip portion 13909b with sufficient sharpness so as to initiate and complete insertion without the need for a separate sharp. In some embodiments, sensor 13900 comprises a sensor tail 13909b having a bottom end with a V-shaped tip portion. Additionally, analyte sensor 13900 further comprises sufficient free length so as to provide the necessary sensor stiffness to facilitate effective insertion into the subject's skin. Stiffness of the sensor is determined by free length of the sensor. For example, an analyte sensor with a shorter free length will be stiffer. Analyte sensor 13900 can include components similar to those of the embodiment described with respect to FIG. 14C.

FIG. 17B illustrates applicator 9150 in cross-section as a user applies a force in a distal direction to housing 9702 (as indicated by the downward arrow). In some embodiments, a predetermined minimum force must be used so that attachment snap 9726 advances past detent 9724. After detent 9724 has been overcome, e.g., snap 9726 is radially displaced, and further depression of housing 9702 with respect to sheath 9704 further causes guide 902, device carrier 9710, and sensor control device 102 to continue to advance from a proximal position towards a distal position towards the skin. In this stage, the distal end of the guide 902 is protruding further distally than the distal surface of sensor control device 102.

Referring next to FIG. 17C, applicator 9150 is depicted as the distal portion of guide 902 and sensor 13900 make contact with the subject's skin surface. Subsequently, the sensor's sharpened tip portion 13909b pierces the skin, while sensor 13900 is supported by guide 902, and inserts a sensor insertion portion of sensor 13900 into the subject's skin, S. In some embodiments, during this phase, the interior surface of proximal portion 9704a of the sheath 9704 remains engaged with the carrier arms 9732 to prevent radial displacement of the arms 9732, and thus maintains retraction spring 946 in a compressed or partially compressed state.

Referring next to FIG. 17D, applicator 9150 is depicted as sensor 13900 reaches its insertion depth, and device carrier 9710 and sensor control device 102 have reached the distal position. In some embodiments, sensor control device 102 includes an adhesive pad that engages the skin surface of the subject at this stage.

According to another aspect of the embodiments, spring retention element 9732 causes retraction spring 946 to expand and retract guide 902 to its retracted position. In some embodiments, for example, spring 946 is a passive element that, during retraction, expands and can return guide 902 to its initial position, or alternatively, be captured by a latch 9733 (FIG. 17A) at some point in between which would allow the guide 902 to be at a different position than its initial position. According to many of the embodiments, sensor 13900 is maintained in its inserted position and sensor control device 102 is left attached to the skin.

Referring still to FIG. 17D, in some embodiments, retraction can be activated when carrier arms 9732 are advanced distally beyond shelf 9704b of sheath and clear a support wall. This allows carrier arms 9732 to deflect radially outwardly into the larger diameter distal portion 9704c of the sheath 9704. When carrier arms 9732 deflect outwardly, shoulder portions of carrier arms 9732 are no longer in an interference relationship with the guide 902, which can be retracted in a proximal direction to its retracted position within applicator 9150. According to another aspect of some embodiments, the retraction step can also be aided by the counterforce created by the skin (and skin deformation), as described earlier with respect to FIG. 16A to 16J.

According to another aspect of some embodiments, housing 9702 is maintained in the distal position by a lock-out feature. In some embodiments, for example, a sheath snap of the sheath 9704 can move up to lock over feature 9722 of the housing 9702. Now the housing 9702 and the sheath 9704 can no longer slidably move with respect to each other, and provides an indication to a user that the inserter has been used.

With respect to retraction spring 946, it should be noted that although compression springs are shown in FIGS. 17A-17D, those of skill in the art will appreciate that other types of springs can be utilized in any of the embodiments described herein, including but not limited to torsion springs, disc springs, leaf springs and others. Furthermore, those of skill in the art will understand that insertion efficiency of the applicator embodiments described herein can be changed by changing insertion speed, sensor length, material or shape, and guide reaction force and fit with sensor during insertion. Additionally, insertion efficiency may be changed by pre-penetrating the skin, as well. Similarly, those of skill in the art will understand that insertion and retraction speeds of the applicator embodiments described herein can be changed by changing the stiffness or length of the retraction spring, and the insertion force and travel length of the sensor.

In some embodiments, the applicator may be a powered applicator and comprise an insertion spring to drive the sensor (and other elements) into the skin of the subject. In the powered applicator embodiment, the insertion spring can be compressed and pre-loaded before it is fired.

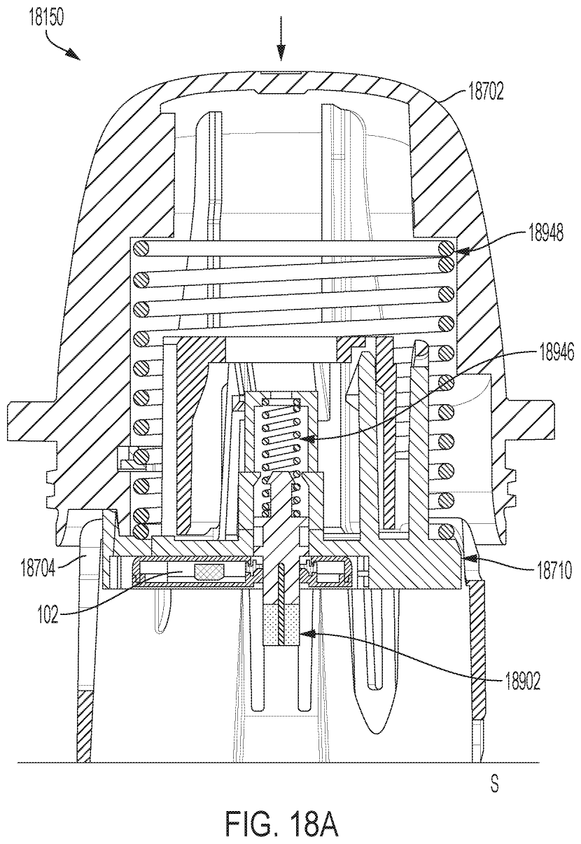

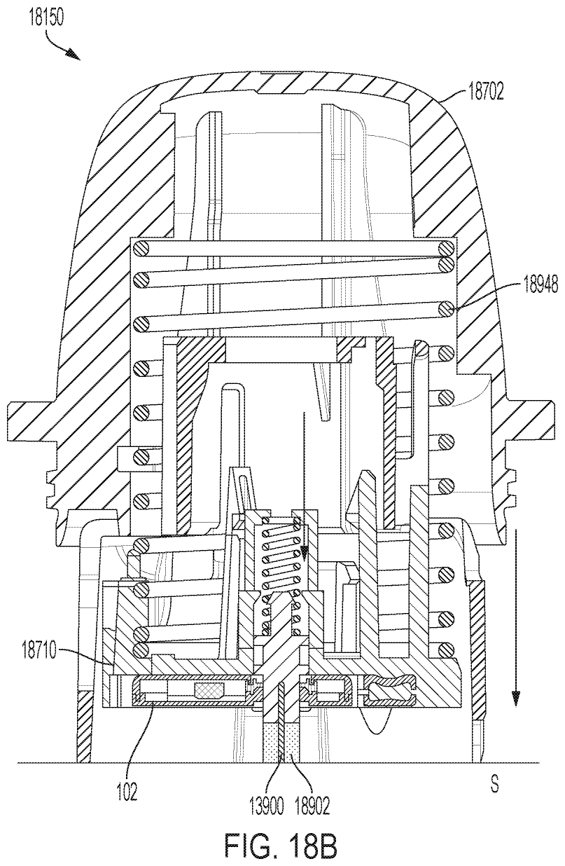

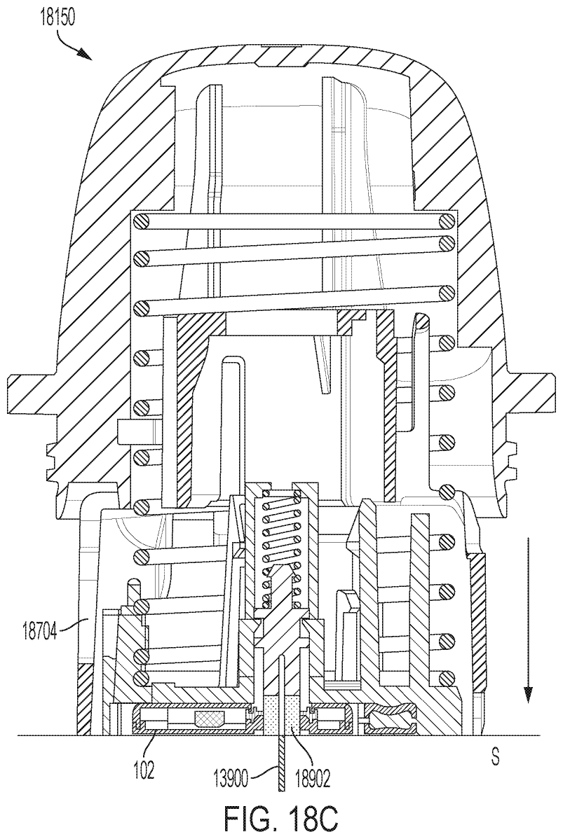

FIGS. 18A to 18C depict cross-sectional views of a powered and sharpless applicator 18150 in various stages of operation. In many respects, applicator 18150 operates in a similar manner to applicator 9150, as described with respect to FIGS. 17A to 17D. For example, applicator 18150 can include many of the same components to applicator 9150, including a housing 18702, sheath 18704, retraction spring 18946, device carrier 18710, and guide 18902, each of which operates in a substantially similar manner to its corresponding component in applicator 9150 (FIGS. 17A to 17D). According to one aspect of the embodiments, however, applicator 18150 further comprises an insertion spring 18948 configured to facilitate a powered (or partially powered) insertion of the sensor.

Referring first to FIG. 18A, applicator 18150 is depicted in an initial stage, where user begins to apply a force upon housing 18702 in a distal direction (as indicated by the downward arrow above applicator 18150). According to one aspect of the embodiments, before the force is applied, carrier 18710, guide 18902, and sensor control device 102 are in a proximal position entirely within applicator 18150. According to another aspect of the embodiments, both insertion spring 18948 and retraction spring 18946 are in a compressed or partially compressed state.

Referring next to FIG. 18B, insertion spring 18948 is shown in an activated state, which causes device carrier 18710, sensor control device 102, and guide 18902 to advance from the proximal position to a distal position (as indicated by the downward arrow to the right of applicator 18150). At this stage, guide 18902 is in contact with the subject's skin, S, and configured to support sensor 13900 during insertion. In some embodiments, insertion spring 18948 is triggered when housing 18702 is advanced a predetermined distance in the distal direction in response to the application of force in the distal direction. For example, housing 18702 can be configured to cause a retention element (not shown) to disengage from insertion spring 18948, causing insertion spring 18948 to expand. In some embodiments, insertion spring 18948 can be activated by a separate button, for example, that can only be depressed if the housing is advanced a predetermined distance in the distal direction. Those of skill in the art will appreciate that other mechanisms for activating insertion spring 18948 can be utilized and are within the scope of the present disclosure.

Referring next to FIG. 18C, sensor 13900 is depicted after it reached the insertion depth (FIG. 18C depicts sensor insertion portion of sensor 13900). At this stage, sensor control device 102 has also reached the distal position. In some embodiments, sensor control device 102 can include an adhesive patch or an adhesive distal-facing surface that is configured to adhere the sensor control device 102 to the subject's skin, S. According to another aspect of the embodiments, retraction spring 18946 has been activated and retracts guide 18902 in the proximal direction to a retracted position. In some embodiments, retracted position of guide 18902 is the same as the initial proximal position of guide 18902, as shown in FIG. 18A. In other embodiments, the retracted position of guide 18902 can be different from the initial proximal position of guide 18902, as shown in FIG. 18A. Subsequently, the applicator 18150 can be removed from the user's skin, S, leaving behind the sensor control device 102 and sensor 13900.

In some embodiments, either or both of sheath 18704 and/or guide 18902 can be locked into the retracted position. As described with respect to FIGS. 17A to 17D, applicator 18150 can include lock-out mechanisms to provide an indication to the user that the applicator has been used.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

1-42. (canceled)

43. An assembly for use with a sensor applicator, the assembly comprising:
a sharp module comprising a sharp portion and a hub portion, wherein the sharp portion comprises a sharp shaft, a sharp proximal end coupled to a distal end of the hub portion, one or more sidewalls that form a sensor channel, and a sharp distal tip configured to penetrate a skin surface of a subject,
an analyte sensor comprising a sensor tail having a sharpened tip portion,
wherein the one or more sidewalls and sensor channel comprise a V-shaped cross-sectional area, the V-shaped cross-sectional area having a vertex.

44. The assembly of claim 43, wherein the one or more sidewalls are disposed along the sharp shaft and comprise one or more beveled edges.

45. The assembly of claim wherein the one or more beveled edges are concavely sloped into the sharp distal tip.

46. The assembly of claim wherein the one or more beveled edges comprise two beveled edges.

47. The assembly of claim 43, wherein a first concavely angled tip portion slopes into a second steep-angled tip portion.

48. The assembly of claim 43, wherein the one or more sidewalls are adjacent to the sharp distal tip.

49. The assembly of claim 43, wherein a terminus of the one or more sidewalls is distal to a terminus of the sensor channel.

50. The assembly of claim 43, wherein a terminus of the one or more sidewalls extends from the vertex.

51. The assembly of claim 43, wherein a terminus of the one or more sidewalls adjoins a proximal portion of the sharp distal tip.

52. The assembly of claim 43, wherein the analyte sensor is an in vivo analyte sensor configured to measure an analyte level in a bodily fluid of the subject.

53. The assembly of claim 43, wherein the sensor tail is in a proximal position relative to the sharp distal tip.

54. The assembly of claim 43, wherein the sensor tail and the sharp distal tip are co-localized.

55. The assembly of claim 43, wherein at least a portion of the analyte sensor is positioned within the sensor channel of the sharp shaft.

56. The assembly of claim 43, wherein the sensor tail has a width sized to fit within the sensor channel.

57. The assembly of claim 43, wherein the sensor tail further comprises an active surface, wherein the active surface is configured to make contact with bodily fluid of the subject.

58. The assembly of claim 43, wherein the sharpened tip portion comprises a V-shaped tip portion.

59. The assembly of claim 43, wherein the sharpened tip portion comprises one or more beveled edges.

60. The assembly of claim 43, wherein the vertex is centrally located in the sensor channel.

61. The assembly of claim 43, wherein a cross-sectional area of the distal tip is less than the V-shaped cross-sectional area of the one or more sidewalls and sensor channel.

62. The assembly of claim 43, wherein a cross-sectional area of the distal tip is the smallest cross-sectional area of the sharp module.

63. The assembly of claim 43, wherein the vertex comprises a bottom portion with no sharp edges.
